(19) United States
(12) Patent Application Publication
(10) Pub. No.: US 2024/0216431 A1
(43) Pub. Date: Jul. 4, 2024

(54) METHODS FOR EXPANSION OR DEPLETION OF T-REGULATORY CELLS

(71) Applicant: The General Hospital Corporation, Boston, MA (US)

(72) Inventor: Denise L. FAUSTMAN, Boston, MA (US)

(21) Appl. No.: 18/540,207

(22) Filed: Dec. 14, 2023

Related U.S. Application Data
(63) Continuation of application No. 16/986,755, filed on Aug. 6, 2020, now Pat. No. 11,844,814.
Application No. 16/986,755 is a continuation of application No. 15/817,713, filed on Nov. 20, 2017, now Pat. No. 10,765,700.
Application No. 15/817,713 is a continuation of application No. 14/764,325, filed on Jul. 29, 2015, now Pat. No. 9,821,010.
(60) Provisional application No. 61/763,217, filed on Feb. 11, 2013.
(60) Provisional application No. 61/762,136, filed on Feb. 7, 2013.

(51) Int. Cl. A61K 35/17; A61K 39/00; C07K 16/28; C12N 5/0783

(52) U.S. Cl. CPC A61K 35/17; A61K 39/00; C07K 16/2878; C12N 5/0637; A61K 2039/515; C07K 2317/34; C07K 2317/73; C07K 2317/74; C07K 2317/75; C07K 2317/76; C07K 2317/92; C12N 2501/04; C12N 2501/2302; C12N 2501/25; C12N 2501/51; C12N 2501/515; C12N 2501/60

(57) ABSTRACT

The invention features methods of producing compositions enriched in Tregs and methods for treating immunological disorders using these compositions. The invention also features methods for producing compositions enriched in lymphocytes and depleted of Tregs and the use of these compositions in the treatment of proliferative disorders.

### SEQUENCE LISTING

The instant application contains a Sequence Listing which has been submitted electronically in XML format and is hereby incorporated by reference in its entirety. Said XML copy, created on Sep. 18, 2023, is named “00786-567007_Sequence_Listing_9_18_23” and is 2,038 bytes in size.

### BACKGROUND OF THE INVENTION

T-regulatory cells (Tregs) are a small subset of T-lymphocytes with diverse clinical applications in transplantation, allergy, asthma, infectious diseases, graft versus host disease (GVHD), and autoimmunity. Tregs are also involved in immunotolerance in conditions such as cancer. The use of Tregs in clinical applications has been challenging because of their rarity in blood and the difficulty of expanding them ex vivo into homogeneous populations. Naturally occurring Tregs constitute only 1-5% of total CD4+ T cells in blood and they remain largely dormant until activated. Therefore, the harvesting of sufficient quantities of Tregs in order to investigate their role in basic biology and for clinical medical applications relies on the ability to expand Tregs ex vivo. More than a dozen protocols have been developed worldwide to expand Tregs ex vivo for reinfusion into patients, but all of these protocols produce heterogeneous progeny consisting of phenotypically and functionally mixed populations of CD4+ T cells. Heterogeneous CD4+ T cell populations hold risk because they are capable of releasing pro-inflammatory cytokines and they possess cells with diverse, sometimes antagonistic functions. Heterogeneous populations of CD4+ T cells are deemed by regulatory agencies to be impure and irreproducible, so no clinical trials have proceeded beyond Phase I studies. Thus, a key research and clinical goal has been to find methods to selectively expand Tregs without stimulating expansion of other CD4+ T cell populations. A parallel goal in this field has been to find methods to selectively deplete Tregs and to expand lymphocyte populations. Such lymphocyte populations would be useful to upregulate the immune response in therapies for proliferative disorders, such as cancers.

### SUMMARY OF THE INVENTION

The invention features a composition enriched in CD4+CD25^{hi} T regulatory cells (Tregs) in which at least 60% (e.g., 70%, 80%, 90%, or 100%) of the cells in the composition are Tregs. Preferably, the composition includes a homogeneous population of Tregs with desirable immune modulating properties, e.g., expression of forkhead box P3 (FOXP3) protein. The composition also includes at least 5×10⁶ (e.g., 5×10⁷, 5×10⁸, 5×10⁹, 5×10¹⁰, 5×10¹¹, or 5×10¹²) Tregs. The Tregs in the composition can be characterized as positive for the expression of one or more proteins selected from the group consisting of CTLA4, TNFR2, FOXP3, CD62L, Fas, HLA-DR, and CD45RO, and as low or negative for the expression of one or more proteins selected from the group consisting of CD127, CCR5, CCR6, CCR7, CXCR3, IFN-gamma, IL10, and ICOS.

The invention also features a method for producing a composition enriched in CD4+CD25^{hi} Tregs, such as the composition described above. This method generally includes contacting in vitro a population of human cells that include T lymphocytes (e.g., CD4+ cells, CD25+ cells, or CD4+CD25+ cells) with a tumor necrosis factor receptor 2 (TNFR2) agonist and/or an NF-κB activator (e.g., during one or more culturing steps), thereby producing a composition that is enriched in the CD4+CD25^{hi} Tregs. The population of human cells can be obtained from a human blood sample or a human bone marrow sample from a patient. The population of human cells from the sample are, or can include, CD4+ cells, CD25+ cells, or CD4+CD25+ cells, which can be isolated or enriched from the blood or bone marrow sample prior to contacting with the TNFR2 agonist and/or the NF-κB activator. The TNFR2 agonist and/or the NF-κB activator promote enrichment of the CD4+CD25^{hi} Tregs, according to the method, by promoting an increase in the proliferation of CD4+CD25^{hi} Tregs present in the population of human cells and/or by increasing the development of CD4+CD25^{hi} Tregs from T lymphocytes (e.g., CD4+ cells, CD25+ cells, or CD4+CD25+ cells) present in the population of human cells (e.g., by differentiation or activation). The method described above preferably produces a homogenous population of Tregs, e.g., where at least 60% (e.g., 70%, 80%, 90%, or substantially 100%) of the cells in the composition are Tregs.

The TNFR2 agonist that can be used in the methods of the invention can be an agent selected from the group consisting of an antibody (e.g., a monoclonal anti-TNFR2 antibody), a peptide, a small molecule, and a protein. Because TNFR2 signaling can proceed via the downstream NF-κB pathway, an NF-κB activator can be used to contact the population of human cells in order to produce the composition enriched in Tregs. The NF-κB activator can be selected from the group consisting of a small molecule (e.g., betulinic acid, topoisomerase poison VP16, and doxorubicin), a peptide, a protein, a virus, and a small non-coding RNA.

In addition to a TNFR2 agonist and/or a NF-κB activator, the method of producing a composition enriched in Tregs can include contacting the population of human cells (e.g., CD4+ cells, CD25+ cells, or CD4+CD25+ cells) with one or more of interleukin-2 (IL2), rapamycin, anti-CD3 (e.g., an anti-CD3 antibody), and/or anti-CD28 (e.g., an anti-CD28 antibody). After in vitro proliferation, the above described methods of the invention can produce at least 5×10⁶ (e.g., 5×10⁶, 5×10⁷, 5×10⁸, 5×10⁹, 5×10¹⁰, 5×10¹¹, or 5×10¹²) Tregs in which at least 60% (e.g., 70%, 80%, 90%, or substantially 100%) of the cells in the composition are Tregs.

The invention also features methods for treating an immunological disorder (e.g., an allergy, asthma, an autoimmune disorder, GVHD, or transplantation graft rejection) or an infectious disease (e.g., a bacterial infection, a viral infection, a fungal infection, and/or a parasitic infection) in a patient (e.g., a human patient) by administering to the patient any one or more of a composition enriched in Tregs, a TNFR2 agonist (e.g., a monoclonal anti-TNFR2 antibody), and a NF-κB activator. For example, the method of treatment can include administering the composition enriched in Tregs by itself or in combination with a NF-κB activator. The composition enriched in Tregs can be produced by any method known in the art. One method of producing a composition enriched in Tregs is by using the methods of the invention described above. The TNFR2 agonist and the NF-κB activator for use in the method of treating an immunological disorder can be any one or more of those described above.

Allergies that can be treated by the methods of the invention can be selected from the group consisting of food allergy, seasonal allergy, pet allergy, hives, hay fever, allergic conjunctivitis, poison ivy allergy oak allergy, mold allergy, drug allergy, dust allergy, cosmetic allergy, and chemical allergy. Autoimmune disorders that can be treated by the methods of the invention can be selected from the group consisting of type I diabetes, Alopecia Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Essential Mixed Cryoglobulinemia, Fibromyalgia-Fibromyositis, Graves' Disease, Guillain-Barré, Hashimoto's Thyroiditis, Hypothyroidism, Idiopathic Pulmonary Fibrosis, Idiopathic Thrombocytopenia Purpura (ITP), IgA Nephropathy, Juvenile Arthritis, Lichen Planus, Lupus, Menière's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiff-Man Syndrome, Takayasu Arteritis, Temporal Arteritis/Giant Cell Arteritis, Ulcerative Colitis, Uveitis, Vasculitis, Vitiligo, and Wegener's Granulomatosis.

The above described methods of treatment can include administering a composition enriched in Tregs that includes at least 5×10⁶ (e.g., 5×10⁶, 5×10⁷, 5×10⁸, 5×10⁹, 5×10¹⁰, 5×10¹¹, or 5×10¹²) Tregs. Tregs having desirable immune-modulating properties include those expressing, e.g., FOXP3.

The invention also features an isolated antibody or antigen-binding fragment thereof that selectively binds to a first epitope of TNFR2, the first epitope includes positions 48-67 of SEQ ID NO: 1. The antibody or antigen-binding fragment thereof has an antagonistic effect on TNFR2 upon binding. The antibody or antigen-binding fragment thereof can further bind to a second epitope of TNFR2. The second epitope includes position 135 of SEQ ID NO: 1. The second epitope can include positions 135-147 of SEQ ID NO: 1 (e.g., positions 130-149 of SEQ ID NO: 1, positions 128-147 of SEQ ID NO: 1, or positions 135-153 of SEQ ID NO: 1). The antibody or antigen-binding fragment thereof can be a monoclonal antibody or antigen-binding fragment thereof, a polyclonal antibody or antigen-binding fragment thereof, an Fab, a humanized antibody or antigen-binding fragment thereof, a bispecific antibody or antigen-binding fragment thereof, a monovalent antibody or antigen-binding fragment thereof, a chimeric antibody or antigen-binding fragment thereof, a single-chain Fv molecule, a bispecific single chain Fv ((scFv′)₂) molecule, a domain antibody, a diabody, a triabody, an affibody, a domain antibody, a SMIP, a nanobody, a Fv fragment, a Fab fragment, a F(ab′)₂ molecule, or a tandem scFv (taFv) fragment. The equilibrium dissociation constant (“K_{D}”) for binding of the antibody or antigen-binding fragment thereof to TNFR2 can be less than about 50 nM (e.g., less than about 30 nM, less than about 20 nM, less than about 10 nM, less than about 5 nM, less than about 2 nM, less than about 1 nM, less than about 900 pM, less than about 800 pM, or less than about 700 pM). The equilibrium dissociation constant (“K_{D}”) for binding of the antibody or antigen-binding fragment thereof to TNFR2 can be in the range of about 10 pM to about 50 nM (e.g., about 20 pM to about 30 nM, about 50 pM to about 20 nM, about 100 pM to about 5 nM, about 150 pM to about 1 nM, or about 200 pM to about 800 pM).

The invention also features a composition enriched in lymphocytes and depleted of Tregs, in which less than 10% (e.g., less than 9%, 8%, 7%, 5%, or 2% or substantially none) of the cells in the composition are Tregs. This composition can be produced by any method known in the art.

Furthermore, the invention also features methods for producing a composition enriched in lymphocytes and depleted of Tregs. This method generally includes contacting in vitro a population of human cells that include Tregs with a tumor necrosis factor receptor 2 (TNFR2) antagonist and/or an NF-κB inhibitor. The TNFR2 antagonist and/or the NF-κB inhibitor is used to suppress the proliferation of Tregs, thereby producing a composition that is substantially depleted of Tregs. The population of human cells can be obtained from a human blood sample or a human bone marrow sample from a patient. The population of human cells can include, e.g., CD4+ cells, CD25+ cells, or CD4+CD25+ cells, which can be isolated or enriched from the blood or bone marrow sample prior to contacting with a TNFR2 antagonist and/or an NF-κB inhibitor. The method described above can be used to produce a composition enriched in lymphocytes (e.g., in which substantially 100% of the cells in the composition are lymphocytes) and in which less than 10% (e.g., less than 9%, 8%, 7%, 5%, or 2% or substantially none) of the cells in the composition are Tregs.

The TNFR2 antagonist that can be used in the above method for producing a composition enriched in lymphocytes and depleted of Tregs can be an agent that is selected from the group consisting of an antibody (e.g., a monoclonal anti-TNFR2 antibody), a peptide, a small molecule, and a protein. The NF-κB inhibitor that can be used in the above method can be an agent selected from the group consisting of a small molecule, a peptide (e.g., a cell penetrating inhibitory peptide), a protein, a virus, and a small non-coding RNA. For example, the NF-κB inhibitor can be a small molecule selected from the group consisting of 2-(1,8-naphthyridin-2-yl)-Phenol, 5-Aminosalicylic acid, BAY 11-7082, BAY 11-7085, CAPE (Caffeic Acid Phenethylester), Diethylmaleate, Ethyl 3,4-Dihydroxycinnamate, Helenalin, Gliotoxin, NF-κB Activation Inhibitor II JSH-23, NFκB Activation Inhibitor III, Glucocorticoid Receptor Modulator, CpdA, PPM-18, Pyrrolidinedithiocarbamic acid ammonium salt, (R)-MG-132, Rocaglamide, Sodium Salicylate, QNZ, MG-132 [Z-Leu-Leu-Leu-CHO], Astaxanthin, (E)-2-Fluoro-4′-methoxystilbene, CHS-828, disulfiram, olmesartan, triptolide, withaferin, celastrol, tanshinone IIA, Ro 106-9920, cardamonin, BAY 11-7821, PSI, HU 211, ML130, PR 39, honokiol, CDI 2858522, andrographolide, and dithiocarbamates.

The TNFR2 antagonist can be a TNFR2 antagonist antibody that binds to a first epitope of TNFR2. The first epitope includes the positions 48-67 of SEQ ID NO: 1. The antibody or antigen-binding fragment thereof can bind to a second epitope of TNFR2. The second epitope includes the position 135 of SEQ ID NO: 1 (e.g., positions 135-147 of SEQ ID NO: 1). The antibody or antigen-binding fragment thereof can be a monoclonal antibody or antigen-binding fragment thereof, a polyclonal antibody or antigen-binding fragment thereof, an Fab, a humanized antibody or antigen-binding fragment thereof, a bispecific antibody or antigen-binding fragment thereof, a monovalent antibody or antigen-binding fragment thereof, a chimeric antibody or antigen-binding fragment thereof, a single-chain Fv molecule, a bispecific single chain Fv ((scFv′)₂) molecule, a domain antibody, a diabody, a triabody, an affibody, a domain antibody, a SMIP, a nanobody, a Fv fragment, a Fab fragment, a F(ab′)₂ molecule, or a tandem scFv (taFv) fragment. The equilibrium dissociation constant (“K_{D}”) for binding of the antibody or antigen-binding fragment thereof to TNFR2 can be less than about 50 nM (e.g., less than about 30 nM, less than about 20 nM, less than about 10 nM, less than about 5 nM, less than about 2 nM, less than about 1 nM, less than about 900 pM, less than about 800 pM, or less than about 700 pM). The equilibrium dissociation constant (“K_{D}”) for binding of the antibody or antigen-binding fragment thereof to TNFR2 can be in the range of about 10 pM to about 50 nM (e.g., about 20 pM to about 30 nM, about 50 PM to about 20 nM, about 100 pM to about 5 nM, about 150 pM to about 1 nM, or about 200 pM to about 800 pM).

The invention features a method of treating a proliferative disorder (e.g., a cancer or a solid tumor) in a patient (e.g., a human patient) by administering to the patient any one or more of a composition enriched in lymphocytes and depleted of Tregs, a TNFR2 antagonist (e.g., a monoclonal anti-TNFR2 antibody), or an NF-κB inhibitor. For example, the method of treating proliferative disorders can include administering the composition enriched in lymphocytes (and depleted of Tregs) by itself or in combination with a NF-κB inhibitor. The composition enriched in lymphocytes can be produced by any method known in the art. Preferably, the composition enriched in lymphocytes can be produced by the methods of the invention as described above. The TNFR2 antagonist and the NF-κB inhibitor for use in the method of treating a proliferative disorder can be any one or more of those described above.

The invention features a method of treating an infectious disease (e.g., a bacterial infection, a viral infection, a fungal infection, or a parasitic infection) in a patient by administering to the patient the composition enriched in lymphocytes and depleted of Tregs, a TNFR2 antagonist (e.g., a monoclonal anti-TNFR2 antibody), or an NF-κB inhibitor. For example, the method of treating an infectious disease can include administering the composition enriched in lymphocytes (and depleted of Tregs) by itself or in combination with a NF-κB inhibitor. The composition enriched in lymphocytes can be produced by any method known in the art. Preferably, the composition enriched in lymphocytes can be produced by the methods of the invention as described above. The TNFR2 antagonist and the NF-κB inhibitor for use in the method of treating a proliferative disorder can be any one or more of those described above.

The invention features a method of treating an infectious disease in a patient by administering to the patient an effective amount of the antibody or antigen-binding fragment thereof as described herein. The invention also features a method of treating a proliferative disease (e.g., a cancer) in a patient by administering to the patient an effective amount of the antibody or antigen-binding fragment thereof as described herein.

Cancers that can be treated according to the methods of the invention (e.g., by administering any one or more of a composition enriched in lymphocytes (and depleted of Tregs), a TNFR2 antagonist and/or an NF-κB inhibitor) can be selected from the group consisting of Acute Lymphoblastic Leukemia, Acute Lymphoblastic Leukemia, Acute Myeloid Leukemia, Adrenocortical Carcinoma; AIDS-Related Lymphoma, AIDS-Related Malignancies, Anal Cancer, Astrocytoma, Bile Duct Cancer, Bladder Cancer; Bone Cancer, Osteosarcoma/Malignant Fibrous Histiocytoma, Brain Stem Glioma, Visual Pathway and Hypothalamic Glioma, Breast Cancer, Bronchial Adenomas/Carcinoids, Chronic Lymphocytic Leukemia, Chronic Myelogenous Leukemia, Chronic Myeloproliferative Disorders, Clear Cell Sarcoma of Tendon Sheaths, Colon Cancer, Colorectal Cancer, Cutaneous T-Cell Lymphoma, Endometrial Cancer, Epithelial Cancer, Esophageal Cancer, Ewing's Family of Tumors, Extracranial Germ Cell Tumor, Extragonadal Germ Cell Tumor, Extrahepatic Bile Duct Cancer, Eye Cancer, Intraocular Melanoma, Retinoblastoma, Gallbladder Cancer, Gastric (Stomach) Cancer, Hairy Cell Leukemia, Head and Neck Cancer, Hepatocellular (Liver) Cancer, Hodgkin's Lymphoma, Hypopharyngeal Cancer, Kaposi's Sarcoma, Kidney Cancer, Laryngeal Cancer, Pituitary Cancer, Plasma Cell Neoplasm/Multiple Myeloma, Pleuropulmonary Blastoma, Skin Cancer, Small Cell Lung Cancer, Small Intestine Cancer, Soft Tissue Sarcoma, Squamous Neck Cancer, Testicular Cancer, Thyroid Cancer, Urethral Cancer, Uterine Sarcoma, and Vaginal Cancer. The solid tumors that can be treated with the methods of the invention can include solid tumors of the brain, lung, breast, lymphoid, gastrointestinal tract, genitourinary tract, pharynx, prostate, or ovary.

### Definitions

The term “about” is used herein to mean a value that is +10% of the recited value.

The term “antibody,” as used herein, includes whole antibodies or immunoglobulins and any antigen-binding fragment or single chains thereof. Antibodies, as used herein, can be mammalian (e.g., human or mouse), humanized, chimeric, recombinant, synthetically produced, or naturally isolated. In most mammals, including humans, whole antibodies have at least two heavy (H) chains and two light (L) chains connected by disulfide bonds. Each heavy chain consists of a heavy chain variable region (abbreviated herein as V_{H}) and a heavy chain constant region. The heavy chain constant region consists of three domains, C_{H}1, C_{H}2, and C_{H}3 and a hinge region between C_{H}1 and C_{H}2. Each light chain consists of a light chain variable region (abbreviated herein as V_{L}) and a light chain constant region. The light chain constant region consists of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. Antibodies of the present invention include all known forms of antibodies and other protein scaffolds with antibody-like properties. For example, the antibody can be a monoclonal antibody, a polyclonal antibody, human antibody, a humanized antibody, a bispecific antibody, a monovalent antibody, a chimeric antibody, or a protein scaffold with antibody-like properties, such as fibronectin or ankyrin repeats. The antibody can have any of the following isotypes: IgG (e.g., IgG1, IgG2, IgG3, and IgG4), IgM, IgA (e.g., IgA1, IgA2, and IgAsec), IgD, or IgE.

The term “antigen-binding fragment,” as used herein, refers to one or more fragments of an antibody that retain the ability to specifically bind to a specific antigen (e.g., CD21 receptor). The antigen-binding function of an antibody can be performed by fragments of a full-length antibody. The antibody fragments can be a Fab, Fab′2, scFv, SMIP, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546, 1989), which consists of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al., Science 242: 423-426, 1988, and Huston et al., Proc. Natl. Acad. Sci. USA 85: 5879-5883, 1988). These antibody fragments are obtained using conventional techniques known to those of skill in the art, and the fragments are screened for utility in the same manner as intact antibodies. Antigen-binding fragments can be produced by recombinant DNA techniques or by enzymatic or chemical cleavage of intact immunoglobulins.

The term “chimeric antibody” refers to an immunoglobulin or antibody whose variable regions derive from a first species and whose constant regions derive from a second species. Chimeric antibodies can be constructed, for example, by genetic engineering, from immunoglobulin gene segments belonging to different species (e.g., from a mouse and a human).

The term “human antibody,” as used herein, is intended to include antibodies, or fragments thereof, having variable regions in which both the framework and CDR regions are derived from human germline immunoglobulin sequences as described, for example, by Kabat et al (Sequences of proteins of Immunological Interest, Fifth Edition, U.S. Department of Health and Human Services, NIH Publication No. 91-3242, 1991). Furthermore, if the antibody contains a constant region, the constant region also is derived from human germline immunoglobulin sequences. The human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences (i.e., a humanized antibody or antibody fragment).

The term “humanized antibody” refers to any antibody or antibody fragment that includes at least one immunoglobulin domain having a variable region that includes a variable framework region substantially derived from a human immunoglobulin or antibody and complementarity determining regions (e.g., at least one CDR) substantially derived from a non-human immunoglobulin or antibody.

The term “TNF-α mutein,” as used herein, refers to a polypeptide having an amino acid sequence that differs from the amino acid sequence of TNF-α by one or more amino acids, while retaining the ability to activate or inhibit TNFR2. For example, a TNF-α mutein may have an amino acid sequence with greater than 90% but less than 100% sequence identity relative to the amino acid sequence of a reference polypeptide (TNF-α).

The term “substantially 100%” or “substantially homogeneous” as used herein with respect to a Treg enriched composition of the invention means at least 90%, 95%, 96%, 97%, 98%, or 99% or more (e.g., all) of the cells in the composition are Tregs.

The term “treating” as used herein means stabilizing or reducing an adverse symptom associated with a condition; reducing the severity of a disease symptom; slowing the rate of the progression of a disease; inhibiting or stabilizing the progression of a disease condition; or changing a metric that is associated with the disease state in a desirable way.

### BRIEF DESCRIPTION OF THE DRAWINGS

FIG. 1A is a set of graphs showing that in a small double-blinded, placebo-controlled trial of human subjects, BCG treatment induces TNF-α (top left graph) and shortly thereafter Tregs appear in the treated subject (bottom left graph) versus placebo (right hand side graphs).

FIG. 1B is a set of graphs showing that in freshly isolated CD4+ cells from fresh human blood, TNF-α alone does not induce FOXP3 in culture (left graph), but does induce it to higher levels when co-incubated with IL-2, compared to IL-2 alone (right graph). The data are from 14 subjects (left panel) and 10 subjects (right panel).

FIG. 1C is a set of representative flow cytometry histograms that confirm greater intracellular induction of FOXP3 in CD4+CD25^{hi} Tregs after co-incubation with TNF-α and IL-2 than with IL-2 alone. Figures in flow diagrams are %. [*P<0.05 or ** P<0.01, by paired t-test].

FIG. 2A is a set of graphs showing that TNFR2 is preferentially expressed on CD4+CD25^{hi} T cells.

FIG. 2B is a graph showing that one TNFR2 antibody induced FOXP3, acting as an agonist, and the other TNFR2 antibody suppressed FOXP3+ expression, acting as antagonist.

FIG. 2C is a graph showing that in a signaling pathway assay, purified CD4+ cells, incubated with IL-2, the TNFR2 agonist and antagonist trigger differences in relative downstream expression of mRNA, especially in signaling proteins TRAF2, TRAF3 and apoptosis inhibitor cIAP2 that were preferentially induced by TNFR2 agonism. Data represented are means±SEM from 4 subjects.

FIG. 2D is a set of graphs showing that TNFR2 agonist triggers greater % increase in proliferation in samples from 6 subjects measured by flow cytometry (left panel) and with carboxyfluorescein diacetate succinimidyl ester (CFSE) measurements (right panels) and representative results from a typical experiment is presented with CFSE measurements (right panels). The numbers in a bar represent the percentage of cells that went into division. The TNFR2 antagonist suppressed CD4+ proliferation (left panel) and inhibited expansion AS measured by CFSE dilution (right panel). * P<0.05 or *** P<0.01, by paired t-test.

FIG. 3A is a schematic showing the protocol for purifying CD4+CD25^{hi} cells from CD4+ cells from fresh blood and expanding for 16 days by incubation in 96 well round-bottom plate (2×10⁴ cells/well) with anti-CD3 and anti-CD28 antibodies, human IL-2, and rapamycin.

FIG. 3B is a set of graphs showing representative CD25 and FOXP3 flow diagrams of CD4+ cells before versus after CD25^{hi} purification and expansion, indicating purity of populations.

FIG. 3C is a graph showing cell counts of purified Tregs, by treatment group, reveal that TNFR2 agonist induced more expansion than any other group. * p<0.05, ** p<0.01, by paired t-test. The TNFR2 antagonist suppressed expansion versus no treatment. Data in FIG. 3C are samples from 10 subjects.

FIG. 4A is a set of graphs showing that all treatment groups are highly positive for Treg markers such as CD25, FOXP3, CTLA4, TNFR2, CD62L, and Fas and negative for CD127.

FIG. 4B is a set of graphs showing that TNFR2 agonist-treated Tregs almost uniformly express HLA-DR and CD45RO and almost uniformly lack markers such as ICOS, CXCR3, CCR5, CCR6, CCR7, and CXCR3.

FIG. 4C is a set of representative flow diagrams showing that TNFR2 agonist-treated Tregs have greater uniformity of Treg markers than do other groups (* p<0.05, ** p<0.01 by t-test).

FIG. 5A is a set of graphs showing that in a representative case, TNFR2 agonist-treated Tregs exerted stronger and dose-dependent suppression of CD8+ cell numbers, compared to other groups, at all dilutions or suppression ratios (left panel, third column). Using a suppression index of 2:1 (CD8+ Responders to Tregs), TNFR2 agonist suppression of CD8+ cells is greater than no treatment and TNFR2 antagonist treatment (right panel). Data in FIG. 5A (right panel) are samples from 5 subjects and data in FIG. 5B (lower panel) are from 8 subjects.

FIG. 5B is a set of graphs showing that TNFR2 agonist-treated Tregs produce a lower percentage of IFNγ+ cells.

FIG. 5C is a graph showing lower numbers of T-bet+ cells after stimulation with PMA and ionomycin for 24 hours.

FIG. 6 is a schematic showing the summary of findings with TNFR2 agonist versus antagonist. After purification and expansion, the TNFR2 agonist is better than TNFR2 antagonist at proliferating and yielding more phenotypically homogeneous Tregs (CD4+CD25^{hi} FOXP3+CTLA4+ TNFR2+CD45RO+CD62L+CD127⁻, HLA-DRhi CCR5− CCR7− CXCR3− ICOS−), with higher suppression capacity for CD8+ cells, and lower cytokine-producing capability.

FIG. 7 is a graph showing the induction of FOXP3 expression by different TNFR agonists and antagonist antibodies. Screening anti-TNFR1 and anti-TNFR2 mAbs reveals that not all of the antibodies tested induce or inhibit FOXP3+ expression.

FIG. 8 is a graph showing the proportion of CD25+FOXP3− cells after IL-2 overnight incubation with and without the TNFR2 agonist or antagonist. Significant percentage increases were observed if the treatment group was incubated in the presence of TNF or TNFR2 agonist. (**; p<0.001). Data are of samples from 10 subjects.

FIG. 9A is a schematic showing the expansion protocol. After 16 days of expansion, Tregs Expander Beads were removed and rested overnight for cell counting.

FIG. 9B is a graph showing the magnitude of expansion by each treatment group. (*; p<0.05, by paired t-test). Data in FIG. 9B are of samples from 10 subjects.

FIG. 10A is a set of graphs showing that all cells were positive for Fas.

FIG. 10B is a set of graphs showing that some surface markers showed diverse patterns of expression according to the way the cells were expanded with a similar trend compared to cells expanded using rapamycin. (*; p<0.05, **; p<0.01) determined by paired t test). Data are from 6 subjects.

FIG. 11A is a graph showing the phenotypes of freshly separated Tregs before expansion (N=3, samples from 3 subjects).

FIG. 11B is a set of graphs showing representative flow diagrams of Tregs markers before expansion.

FIG. 12 is a set of graphs showing the density of cell surface markers measured by Mean Fluorescence Intensity (MFI). MFI of Tregs demonstrates clear differences between TNFR2 agonist expanded cells and TNFR2 antagonist expanded cells. (* p<0.05, ** p<0.01 determined by paired t test).

FIG. 13A is a graph showing that the suppression capacity of expanded CD4+CD25+ cells was determined by CFSE dilution of CD8+ T responder cells. Flow cytometric figures of a typical result and summary of suppression index calculated based upon Responder: Treg of 2:1 from four independent experiments is also shown in FIG. 13A.

FIG. 13B is a set of graphs showing that CD4+CD25+ cells expanded with TNFR2 agonist exhibited significant enhanced suppression capacity (N=5). Those cells showed lowest cytokine producing capacity. (IFN, IL-10 and TNF after stimulation with PMA and ionomycin for 24 hours (*; p<0.05, **; p<0.01, by paired t test).

### DETAILED DESCRIPTION OF THE INVENTION

The invention features methods for the production of a composition enriched in Tregs (e.g., CD4+, CD25^{hi} Tregs). The invention also features methods for treating immunological disorders and infectious diseases using a composition enriched in Tregs, e.g., a composition enriched in Tregs prepared by the methods described below. The invention also features methods for producing a composition enriched in lymphocytes (and depleted of Tregs) and methods of treating proliferative disorders using this composition.

### Tregs and TNFR2

T regulatory cells (Tregs) are a small subset of T-lymphocytes with diverse clinical applications in transplantation, allergy, asthma, infectious diseases, GVHD, and autoimmunity. The Tregs can be used to suppress the abnormal immune response in patients in need thereof. Tregs are also known to be involved in immunotolerance in conditions such as cancer. Naturally occurring Tregs constitute only 1-5% of total CD4+ T cells in blood, and remain largely dormant until activated. In humans, Tregs are defined by co-expression of CD4+ and high expression of the interleukin-2 (IL-2) receptor alpha chain CD25^{hi}. Tregs also feature inducible levels of intracellular transcription factor FOXP3 and the expression of FOXP3 can be used to identify Tregs. TNF-α has two receptors, TNFR1 and TNFR2, each of which controls different signaling pathways. Unlike TNFR1, which has ubiquitous cellular expression, TNFR2 is expressed in a more limited manner, restricted primarily to subpopulations of T cells (in particular, Tregs), endothelial cells, and neurons. Research in primates suggests that TNFR2-specific ligands are likely to have minimal systemic toxicity because of the restricted cellular distribution of TNFR2. Naturally occurring Tregs appear to express TNFR2 at a higher density than TNFR1. These features make TNFR2 an advantageous molecular target on Tregs.

### Methods for Producing an Enriched Treg Composition

The invention features methods for expanding Tregs in a sample isolated from a patient (e.g., a human), such as a peripheral blood sample or a bone marrow sample, to produce a composition enriched in Tregs that are characterized as CD4+ and CD25^{hi}. Methods for promoting the proliferation of Tregs are known in the art, e.g., as described in Brunstein, C. G. et al., Infusion of ex vivo expanded T regulatory cells in adults transplanted with umbilical cord blood: safety profile and detection kinetics, Blood 117, 1061-1070 (2011); Saas, P. & Perruche, S., (F1000 Immunology, 2012), Tresoldi, E. et al., Stability of human rapamycin-expanded CD4+CD25+ T regulatory cells, Haematologica 96, 1357-1365 (2011); Nadig, S. N. et al., In vivo prevention of transplant arteriosclerosis by ex vivo-expanded human regulatory T cells. Nat Med 16, 809-813 (2010); Battaglia, M., Stabilini, A. & Tresoldi, E., Expanding human T regulatory cells with the mTOR-inhibitor rapamycin, Methods Mol Biol 821, 279-293 (2012); Pahwa, R. et al., Isolation and expansion of human natural T regulatory cells for cellular therapy, Journal of immunological methods 363, 67-79 (2010); Hoffmann, P., Eder, R., Kunz-Schughart, L. A., Andreesen, R. & Edinger, M., Large-scale in vitro expansion of polyclonal human CD4(+)CD25high regulatory T cells, Blood 104, 895-903 (2004); Lin, C. H. & Hunig, T., Efficient expansion of regulatory T cells in vitro and in vivo with a CD28 superagonist, European journal of immunology 33, 626-638 (2003); Lan, Q. et al., Induced FOXP3(+) regulatory T cells: a potential new weapon to treat autoimmune and inflammatory diseases? Journal of molecular cell biology 4, 22-28 (2012); Sagoo, P. et al., Human regulatory T cells with alloantigen specificity are more potent inhibitors of alloimmune skin graft damage than polyclonal regulatory T cells, Science Translational Medicine 83, 1-10 (2011); Edinger, M. & Hoffmann, P., Regulatory T cells in stem cell transplantation: strategies and first clinical experiences, Current opinion in immunology 23, 679-684 (2011); Trzonkowski, P. et al., First-in-man clinical results of the treatment of patients with graft versus host disease with human ex vivo expanded CD4+CD25+CD127-T regulatory cells, Clinical Immunology 133, 22-26 (2009); Di lanni, M. et al., Tregs prevent GVHD and promote immune reconstitution in HLA-haploidentical transplantation, Blood 117, 3921-3928 (2011); Hippen, K. L. et al, Massive ex vivo expansion of human natural regulatory T cells (T(regs)) with minimal loss of in vivo functional activity. Sci Transl Med 3, 83ra41 (2011); Kim, Y. C. et al., Oligodeoxynucleotides stabilize Helios-expressing Foxp3+ human T regulatory cells during in vitro expansion, Blood 119, 2810-2818 (2012); Bacchetta, R. et al., Interleukin-10 Anergized Donor T Cell Infusion Improves Immune Reconstitution without Severe Graft-Versus-Host-Disease After Haploidentical Hematopoietic Stem Cell Transplantation. ASH Annual Meeting Abstracts 114, 45-(2009); Desreumaux, P. et al., Safety and efficacy of antigen-specific regulatory T-cell therapy for patients with refractory Crohn's disease, Gastroenterology 143, 1207-1217 e1202 (2012); Clerget-Chossat, N. et al., in International Society for Cell Therapy Seattle, Washington, (2012); and Cardenas, P. A., Huang, Y. & Ildstad, S. T., The role of pDC, recipient T(reg) and donor T(reg) in HSC engraftment: Mechanisms of facilitation, Chimerism 2, 65-70 (2011). Each of these publications, and their methods for expanding Tregs, is incorporated herein by reference.

The protocols for promoting the proliferation of Tregs that are described in the above publications generally include obtaining fresh sample (e.g., a blood sample) from a patient (e.g., a human patient) that includes a population of CD4+ cells. The CD4+ cells can be further purified or enriched in one or more steps prior to the expansion of Tregs. The CD4+ cells can be separated from the sample using techniques known in the art (e.g., using magnetic beads conjugated to anti-CD4+ antibodies such as DYNABEADS® CD4 Positive Isolation kit (Invitrogen)). During culturing, the CD4+ cells can be contacted with one or more reagents to stimulate their proliferation. For example, one or more of anti-CD3 antibody, anti-CD28 antibody, human IL-2, and rapamycin can be added. However, this method alone produces a heterogeneous population of cells, some of which are capable of releasing pro-inflammatory cytokines that can be detrimental to the patient. This heterogeneous population is not useful for treatment of immunological disorders or infectious diseases and Tregs cannot be easily isolated from this heterogeneous population without damaging them. The present invention improves upon these protocols by using a TNFR2 agonist that preferentially promotes proliferation of Tregs and produces a homogeneous population of Tregs with desirable traits, e.g., a sub-population of Tregs that express FOXP3. The TNFR2 agonist and/or the NF-κB activator promote enrichment of the CD4+CD25^{hi} Tregs, according to the method, by promoting an increase in the proliferation of CD4+CD25^{hi} Tregs present in the population of human cells and/or by increasing the development of CD4+CD25^{hi} Tregs from T lymphocytes (e.g., CD4+ cells, CD25+ cells, or CD4+CD25+ cells) present in the population of human cells (e.g., by differentiation or activation). The invention produces a population of cells enriched in Tregs that can be used for treatment of immunological disorders or infectious diseases as described herein.

In vitro Expansion of Tregs Using a TNFR2 Agonist

In general, the present method includes obtaining a starting population of T lymphocytes (e.g., CD4+ cells, CD25+ cells, or CD4+CD25+ cells) from a human sample, e.g., blood or bone marrow sample. When blood sample is used, typically 3-4 tubes of blood (˜2-10 mL in each tube) can be used in the protocol described below. One skilled in the art can adjust the amount of the blood sample that is used depending on the scale of the cell-culture protocol.

In general, anti-CD4 and/or anti-CD25 antibodies attached to a bead matrix, e.g., a magnetic bead (such as DYNABEADS®), can be used for isolating the CD4+ cells, CD25+ cells, or CD4+CD25+ cells. For example, the CD4+ T cells can be isolated by using commercially available reagents, e.g., DYNABEADS® CD4 Positive Isolation kit, and the CD25+ cells can be isolated using commercially available reagents, e.g., DYNABEADS® CD25 and/or DETACHABEAD™ CD4/CD8 (Invitrogen). When CD4+CD25+ cells are used as the starting population, the cells can be isolated using both anti-CD4 and anti-CD25 beads in a single step or in a two step method by isolating the CD4+ cells first followed by isolation of the CD25+ cells, or vice versa.

The isolated CD4+ cells, CD25+ cells, or CD4+CD25+ cells can then be expanded in cell culture for about 16 days (e.g., about 5, 6, 7, 8, 9, 10, 11, 12, 13, 14, 15, 16, 17, or 18 or more days) in a suitable cell culture vessel, e.g., a 96 well round-bottom plate (2×10⁴ cells/well), in the presence of one or more of anti-CD3 and/or anti-CD28 antibodies. The amount of cells to be used in the starting culture will depend on the volume of the cell culture vessel. The anti-CD3 and anti-CD28 antibodies can be present throughout the course of cell culture, e.g., for each day of culture or only during a portion of the cell culture (one or several days during culturing). Typically the anti-CD3 and anti-CD28 antibodies can be added in the form of commercially available DYNABEADS® Human Treg Expander (Invitrogen) at a bead to cell ratio of 2:1.

Human IL-2 and/or rapamycin can also be added to the cell culture media, e.g., one or more times during the course of cell culture. For example, human IL-2 can be added every two days, e.g., at day 2, 4, 7, 9, 11, and 14 of a 16-day cell culture period. Rapamycin can be added at day 0, 2, 4, and 7 of a 16 day-cell culture period. Human IL-2 and rapamycin can be added together or on alternating days. Typically, human IL-2 is added two days after the start of cell culture. Human IL-2 and/or rapamycin can also remain in the cell culture for the entire period of the expansion protocol. The cell culture media can be changed every 2-3 days by changing half of the media with fresh media; the fresh media may also contain human IL-2 and/or rapamycin. For example, half of the media can be changed every 2-3 days containing rapamycin (until day 7) and IL-2. Typically rapamycin can be used at a concentration of 0.5 nM to 100 μM (e.g., 0.5 nM, 1 nM, 10 nM, 50 nM, 100 nM, 200 nM, 0.5 μM, 0.75 μM, 1 μM, 1.2 μM, or 2 μM). and human IL-2 can be used at a concentration of 0.05 to 6,000 U/ml (e.g., 0.05 U/ml, 1 U/ml, 2 U/ml, 10 U/ml, 20 U/ml, 50 U/ml, 100 U/ml, 150 U/ml, 200 U/ml, 250 U/ml, or 300 U/ml). During the course of the cell culture, as described above, the cells can be passaged as necessary. Protocols for passaging of cells are known in the art.

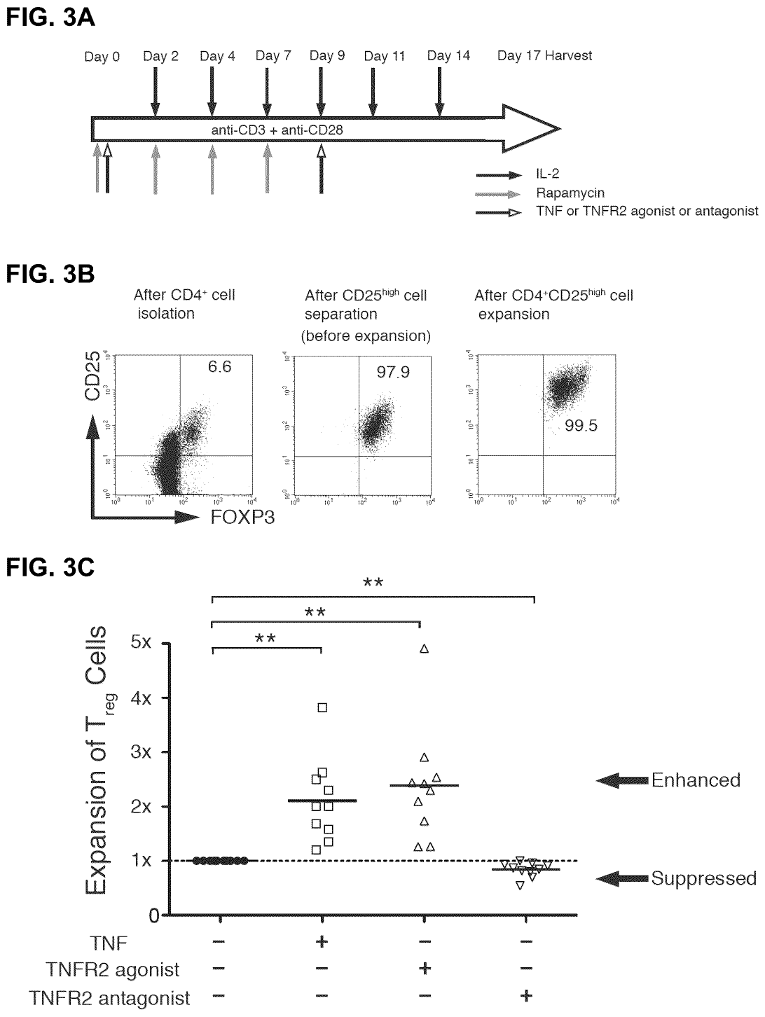

A TNFR2 agonist can be added at the start of culture at day 0 or at later time points after initiation of the culture (e.g., on any one of the days after initiation of culturing, so long as at least one or more days (e.g., at least 2, 3, 4, 5, 6, 7, 8, 9, 10, 11, 12, 13, 14, 15, or 16, or more days) of culturing includes contacting the cells with a TNFR2 agonist). Additional TNFR2 agonists can be added at several points during the course of cell culture, e.g., at one or more of days 7, 8, 9, 10, 11, or 12. Typically a TNFR2 agonist is added on day 0 and additional TNFR2 agonist can be added on day 9 of a 16 day-cell culture period (see, e.g., FIG. 3A). The TNFR2 agonist that can be typically used is an anti-TNFR2 monoclonal antibody. Additional TNFR2 agonists that can be used in this method are described below. In general, the anti-TNFR2 antibody can be used at a concentration in the range of 0.05 μg/ml to 500 μg/ml, or more if necessary (e.g., 0.05 μg/ml, 0.1 μg/ml, 0.25 μg/ml, 0.5 g/ml, 1 μg/ml, 1.5 μg/ml, 2 μg/ml, 2.5 μg/ml, 3 μg/ml, 3.5 μg/ml, 4 g/ml, 4.5 μg/ml, or 5 μg/ml, 10 μg/ml, 50 μg/ml, 100 μg/ml, 200 μg/ml, 300 μg/ml, 400 μg/ml, or 500 μg/ml). The anti-TNFR2 antibody can be attached to a matrix, e.g., a bead, such as a magnetic bead, for removal at the end of the cell culture period.

The cells can be harvested and the anti-CD3 and anti-CD28 regents can be removed, e.g., by removal of the Treg Expander beads by the DETACHABEAD™ reagent or by multiple rounds of proliferation that permits bead detachment. Cells can then be washed in an appropriate medium and rested. Cells can then be analyzed for expression of various protein markers, stored appropriately, and/or used in methods for treating various disorders as described below.

After in vitro proliferation, the Tregs in the enriched composition comprise at least 60% (e.g., 70%, 80%, 90%, or 100%) of the cells in the composition. The method described above preferably produces a homogenous population of Tregs, e.g., where substantially 100% (e.g., at least 90%, 95%, 96%, 97%, 98%, or 99% or more (e.g., all)) of the cells in the composition are Tregs.

The method described above can result in an approximately 2-fold (e.g., 1.5-fold, 2-fold, 2.5-fold, 3-fold, 3.5-fold, or 4-fold or more) expansion of Tregs. The Tregs produced by this expansion protocol are characterized as expressing FOXP3, e.g., preferably at least 80% (e.g., 85%, 90%, 95%, 98%, 99%, or substantially 100%) of the cells in the expanded population of Tregs express FOXP3. In addition, it is preferable that the Tregs expanded by the methods of the invention express high levels of FOXP3. The Treg population also includes a low percentage of cells expressing IFNγ. The Treg population also exhibits increased capacity for suppressing activation of CD8+ cells.

In previously described Treg expansion protocols, a disadvantage was that the identification of the expanded Tregs in a heterogeneous population of cells required post-expansion sorting, often multiple rounds of sorting, of the Tregs. These multi-sorting procedures severely and adversely affected the viability, function, and yield of the Tregs and therefore limited the subsequent use of the sorted cells in therapeutic applications. Furthermore, this sorting could only enrich for Tregs expressing cell surface markers and not for Tregs expressing FOXP3, which is an intracellular marker. The present invention features the proliferation of Tregs by contacting a population of human cells that is, or that includes, T lymphocytes (e.g., human CD4+ cells, CD25+ cells, or CD4+CD25+ cells) with a TNFR2 agonist to produce a substantially homogenous population of Tregs that express FOXP3, CD4+, and CD25^{hi}. The Tregs produced by the present method require no post-expansion sorting prior to use in therapeutic applications. This is a significant advantage over previously described Treg expansion protocols. The Tregs produced by the present invention are also more potent than previously described Treg cell populations, which may be a result of their homogeneity or the subset of Tregs produced by the present method, or both. The present enriched Treg populations exhibit highly desirable qualities similar to those of immune-modulating Tregs.

### TNFR2 Agonists

The TNFR2 agonist that can be used in the methods of the invention include agents, such as an antibody, a peptide, a small molecule, and a protein. The TNFR2 agonist is an agent that can bind to TNFR2 and activate TNFR2 signaling. The TNFR2 agonist can be any agent that, when contacted with CD4+ T cells, can stimulate the expression of any one or more proteins selected from the group consisting of FOXP3, TNF, TRAF2, TRAF3, and cIAP2.

In particular, the TNFR2 agonist can be a monoclonal antibody that binds TNFR2, such as Clone MR2-1 (Cell Sciences) or Clone MAB2261 (R&D Systems, Inc.). The TNFR2 agonist can also be a TNF-α mutein that binds only to TNFR2 as an agonist. TNF-α muteins that can be used as TNFR2 agonists include those described in, e.g., U.S. Patent Application Publication No. 2008/0176796 A1; U.S. Pat. Nos. 5,486,463 and 5,422,104; PCT Publication Nos. WO 86/02381; WO 86/04606; and WO 88/06625; and European Patent Nos. 155,549; 168,214; 251,037; 340,333; and 486,908. Each of these publications is incorporated herein by reference.

In addition, anti-TNFR2 antibodies that are capable of acting as TNFR2 agonists are described in Galloway et al. (Eur. J. Immunol. 22:3045-3048, 1992), Tartaglia et al. (J. Biol. Chem. 268:18542-18548, 1993), Tartaglia et al. (J. Immunol. 151:4637-4641, 1993), Smith et al. (J. Biol. Chem. 269:9898-9905, 1994), and Amrani et al. (Am. J. Respir. Cell. Mol. Biol. 15:55-63, 1996); each of which is incorporated herein by reference.

Peptides that are capable of acting as a TNFR2 agonist can include an 11 amino acid TNF receptor agonist peptide (TNF₇₀₋₈₀) described in Laichalk et al. (Infection & Immunity 66:2822-2826, 1998), incorporated herein by reference.

Since activation of the NF-κB pathway is a downstream effect of TNFR2 agonism, the Treg expansion method can instead, or in addition, include contacting the T lymphocyte population (e.g., CD4+ cells, CD25+ cells, or CD4+CD25+ cells) with one or more activators of the NF-κB pathway in place of a TNFR2 agonist. The NF-κB activator can be a small molecule, a peptide, a protein, a virus, or a small non-coding RNA. For example, the NF-κB activator any one of the small molecules described in Manuvakhova et al., J. Neurosci. Res. 89: 58-72, 2011 (incorporated herein by reference). Alternatively, the NF-κB activator can be betulinic acid. The NF-κB activator can also be the topoisomerase poison VP16. Additionally the NF-κB activator can be doxorubicin.

### Characterization of Tregs

The Tregs in the enriched composition produced by the above described methods are CD4+ and CD25^{hi} and can be characterized by the presence or absence of one or more additional molecular markers. For example, the Tregs produced by the methods of the invention may express one or more proteins selected from the group consisting of FOXP3, CTLA4, TNFR2, CD62L, Fas, HLA-DR, and CD45RO and are considered to be “positive” for these markers. Alternatively, the Tregs may not express, or may express in low to near undetectable amounts, one or more proteins selected from the group consisting of CD127, CCR5, CCR6, CCR7, CXCR3, IFN-gamma, IL10, and ICOS and may be considered to be considered “negative” for these markers. Preferably, the method produces a composition enriched in Tregs, in which at least 90% of Tregs express HLA-DR and less than 5% of Tregs express ICOS.

### Treatment Using an Enriched Treg Composition of the Invention

The invention features methods for treating a variety of diseases, e.g., immunological disorders and conditions, such as allergies, asthma, autoimmune diseases, GVHD, transplantation graft rejection, and infectious diseases by administering a composition enriched in Tregs to a patient (e.g., a human) in need thereof. The composition enriched in Tregs can be produced by the methods described above, for example, by contacting a human sample, e.g., a blood or bone marrow sample, containing T lymphocytes (e.g., CD4+ cells, CD25+ cells, or CD4+CD25+ cells) with a TNFR2 agonist (e.g., a TNFR2 agonist antibody) and/or an NF-κB activator to produce the composition enriched in Tregs (e.g., a substantially homogenous population of Tregs). The TNFR2 agonist and/or the NF-κB activator promote enrichment of the CD4+CD25^{hi} Tregs, according to the method, by promoting an increase in the proliferation of CD4+CD25^{hi} Tregs present in the population of human cells and/or by increasing the development of CD4+CD25^{hi} Tregs from T lymphocytes (e.g., CD4+ cells, CD25+ cells, or CD4+CD25+ cells) present in the population of human cells (e.g., by differentiation or activation).

A patient in need of treatment for an immunological disorder or condition, such as an allergy, asthma, an autoimmune disease, GVHD, or a transplantation graft rejection, or for an infectious disease can be administered an enriched composition of Tregs (e.g., CD4+CD25^{hi} Tregs or CD4+CD25^{hi}FOXP3+ Tregs) produced from their own blood or bone marrow using the following steps: i) obtaining a cell sample, e.g., a blood or bone marrow sample, from the human patient; ii) isolating T lymphocytes (e.g., CD4+ cells, CD25+ cells, or CD4+CD25+ cells) from the sample as described in the method above; iii) subjecting these cells to the Tregs expansion method described above (e.g., contact and/or culturing of the CD4+ cells, CD25+ cells, or CD4+CD25+ cells with anti-CD3 antibody, anti-CD28 antibody, and a TNFR2 agonist in combination with IL-2 and/or rapamycin, and/or contact or culturing of the CD4+ cells, CD25+ cells, or CD4+CD25+ cells with an NF-κB activator) to produce a composition enriched in Tregs (e.g., a substantially homogenous population of Tregs in which the Tregs comprise, e.g., >90% of cells in the composition); and iv) introducing the composition enriched in Tregs into the patient without any (or with limited) post-expansion sorting of the enriched Tregs in order to treat the disease or disorder. The above steps of the method of treatment can be performed in iterative cycles, where the number of cycles of treatment provided to the patient can be determined by the disorder being treated, the severity of the disorder, and/or the outcome of each treatment cycle (i.e., a change in the disease state). For example, changes in efficacy markers and/or in clinical outcomes can be used for determining the frequency with which blood or bone marrow should be obtained from a patient, Tregs enriched from that blood or bone marrow, and/or the enriched Tregs administered to the patient. The enriched Treg composition can also be stored (e.g., frozen) for future administration.

Preferably, the Tregs are obtained from a patient's own blood or bone marrow (i.e., autologous cells), although the following methods of treatment may also include the use of allogeneic Tregs with possible best fit HLA matching or from unrelated donors that have been expanded according to the above methods. Allogeneic Tregs preferentially share at least 4/6 HLA markers in common with the patient receiving the enriched Treg composition.

### Treatment of an Immunological Disorder or Condition Using an Enriched Treg Composition of the Invention

The enriched Treg composition produced by the methods described above can be administered to a patient suffering from an immunological disorder or condition, such as an allergy, asthma, an autoimmune disorder, GVHD, or a transplantation graft rejection, to treat the immunological disorder or condition.

### 1) Allergies:

An enriched Treg composition of the invention can be used to treat one or more allergic conditions in a patient, such as an allergy selected from the group consisting of food allergy, seasonal allergy, pet allergy, hives, hay fever, allergic conjunctivitis, poison ivy allergy oak allergy, mold allergy, drug allergy, dust allergy, cosmetic allergy, and chemical allergy. Administration and dosage of the Treg composition are discussed herein below.

### 2) Asthma:

An enriched Treg composition of the invention can be used to treat asthma by administering the composition to a patient in need thereof.

### 3) Autoimmune Disorders:

An enriched Treg composition of the invention can be used to treat one or more autoimmune disorder selected from the group consisting of type I diabetes, Alopecia Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Essential Mixed Cryoglobulinemia, Fibromyalgia-Fibromyositis, Graves' Disease, Guillain-Barre, Hashimoto's Thyroiditis, Hypothyroidism, Idiopathic Pulmonary Fibrosis, Idiopathic Thrombocytopenia Purpura (ITP), IgA Nephropathy, Juvenile Arthritis, Lichen Planus, Lupus, Ménière's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjogren's Syndrome, Stiff-Man Syndrome, Takayasu Arteritis, Temporal Arteritis/Giant Cell Arteritis, Ulcerative Colitis, Uveitis, Vasculitis, Vitiligo, and Wegener's Granulomatosis. Additional autoimmune diseases that can be treated with the methods of the invention are disclosed in U.S. Pat. No. 8,173,129, incorporated herein by reference. Administration and dosage of the Treg composition are discussed herein below.

### 4) Transplantation Graft Rejection or GVHD:

An enriched Treg composition of the invention can be used to reduce or inhibit transplantation graft rejection or GVHD that occurs when transplanted tissue is rejected by the recipient's immune system. The transplantation graft rejection can be a chronic rejection, an acute rejection, or a hyperacute rejection. Administration and dosage of the Treg composition are discussed herein below.

In addition to the composition enriched in Tregs, other treatments that can be administered to the patient can include, e.g., steroid treatment, antibody-based treatment, immosuppressive drugs, blood transfer, and marrow transplant, according to techniques known in the art.

### Treatment of an Infectious Disease Using an Enriched Treg Composition of the Invention

The invention also features methods of treating infectious diseases caused any one or more of a virus, a bacteria, a fungus, or a parasite by administering a composition enriched in Tregs. The composition enriched in Tregs can be produced by the methods described above. The methods of the invention can be used for treating viral infections caused by, e.g., a member of the Flaviviridae family (e.g., a member of the Flavivirus, Pestivirus, and Hepacivirus genera), which includes the hepatitis C virus, Yellow fever virus; Tick-borne viruses, such as the Gadgets Gully virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus and the Negishi virus; seabird tick-borne viruses, such as the Meaban virus, Saumarez Reef virus, and the Tyuleniy virus; mosquito-borne viruses, such as the Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus; and viruses with no known arthropod vector, such as the Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, and the Cell fusing agent virus; a member of the Arenaviridae family, which includes the Ippy virus, Lassa virus (e.g., the Josiah, LP, or GA391 strain), lymphocytic choriomeningitis virus (LCMV), Mobala virus, Mopeia virus, Amapari virus, Flexal virus, Guanarito virus, Junin virus, Latino virus, Machupo virus, Oliveros virus, Paraná virus, Pichinde virus, Pirital virus, Sabia virus, Tacaribe virus, Tamiami virus, Whitewater Arroyo virus, Chapare virus, and Lujo virus; a member of the Bunyaviridae family (e.g., a member of the Hantavirus, Nairovirus, Orthobunyavirus, and Phlebovirus genera), which includes the Hantaan virus, Sin Nombre virus, Dugbe virus, Bunyamwera virus, Rift Valley fever virus, La Crosse virus, California encephalitis virus, and Crimean-Congo hemorrhagic fever (CCHF) virus; a member of the Filoviridae family, which includes the Ebola virus (e.g., the Zaire, Sudan, Ivory Coast, Reston, and Uganda strains) and the Marburg virus (e.g., the Angola, Ci67, Musoke, Popp, Ravn and Lake Victoria strains); a member of the Togaviridae family (e.g., a member of the Alphavirus genus), which includes the Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O'nyong'nyong virus, and the chikungunya virus; a member of the Poxviridae family (e.g., a member of the Orthopoxvirus genus), which includes the smallpox virus, monkeypox virus, and vaccinia virus; a member of the Herpesviridae family, which includes the herpes simplex virus (HSV; types 1, 2, and 6), human herpes virus (e.g., types 7 and 8), cytomegalovirus (CMV), Epstein-Barr virus (EBV), Varicella-Zoster virus, and Kaposi's sarcoma associated-herpesvirus (KSHV); a member of the Orthomyxoviridae family, which includes the influenza virus (A, B, and C), such as the H5N1 avian influenza virus or H1N1 swine flu; a member of the Coronaviridae family, which includes the severe acute respiratory syndrome (SARS) virus; a member of the Rhabdoviridae family, which includes the rabies virus and vesicular stomatitis virus (VSV); a member of the Paramyxoviridae family, which includes the human respiratory syncytial virus (RSV), Newcastle disease virus, hendravirus, nipahvirus, measles virus, rinderpest virus, canine distemper virus, Sendai virus, human parainfluenza virus (e.g., 1, 2, 3, and 4), rhinovirus, and mumps virus; a member of the Picornaviridae family, which includes the poliovirus, human enterovirus (A, B, C, and D), hepatitis A virus, and the coxsackievirus; a member of the Hepadnaviridae family, which includes the hepatitis B virus; a member of the Papillamoviridae family, which includes the human papilloma virus; a member of the Parvoviridae family, which includes the adeno-associated virus; a member of the Astroviridae family, which includes the astrovirus; a member of the Polyomaviridae family, which includes the JC virus, BK virus, and SV40 virus; a member of the Calciviridae family, which includes the Norwalk virus; a member of the Reoviridae family, which includes the rotavirus; and a member of the Retroviridae family, which includes the human immunodeficiency virus (HIV; e.g., types 1 and 2), and human T-lymphotropic virus Types I and II (HTLV-1 and HTLV-2, respectively)).

The methods of the invention can also be used for treating bacterial infections. Examples of bacterial infections that may be treated include, but are not limited to, those caused by bacteria within the genera Salmonella, Streptococcus, Bacillus, Listeria, Corynebacterium, Nocardia, Neisseria, Actinobacter, Moraxella, Enterobacteriacece, Pseudomonas, Escherichia, Klebsiella, Serratia, Enterobacter, Proteus, Salmonella, Shigella, Yersinia, Haemophilus, Bordatella, Legionella, Pasteurella, Francisella, Brucella, Bartonella, Clostridium, Vibrio, Campylobacter, and Staphylococcus.

The methods of the invention can also be used for treating parasitic infections caused by a protozoan parasite (e.g., an intestinal protozoa, a tissue protozoa, or a blood protozoa) or a helminthic parasite (e.g., a nematode, a helminth, an adenophorea, a secementea, a trematode, a fluke (blood flukes, liver flukes, intestinal flukes, and lung flukes), or a cestode). Exemplary protozoan parasites include Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Toxoplasma gondii, Plasmodium vivax, Plasmodium ovale, Plasmodium malariae, Plasmodium falciparum, Trichomonas vaginalis, and Histomonas meleagridis. Exemplary helminthic parasites include richuris trichiura, Ascaris lumbricoides, Enterobius vermicularis, Ancylostoma duodenale, Necator americanus, Strongyloides stercoralis, Wuchereria bancrofti, and Dracunculus medinensis, Schistosoma mansoni, Schistosoma haematobium, Schistosoma japonicum, Fasciola hepatica, Fasciola gigantica, Heterophyes heterophyes, and Paragonimus westermani, Taenia solium, Taenia saginata, Hymenolepis nana, and Echinococcus granulosus.

The methods of the invention can also be used for treating fungal infections. Examples of fungal infections that may be treated include, but are not limited to, those caused by, e.g., Aspergillus, Candida, Malassezia, Trichosporon, Fusarium, Acremonium, Rhizopus, Mucor, Pneumocystis, and Absidia.

Administration and dosage of the Treg composition in methods for treating an infectious disease are discussed herein below.

### Treatment Using an NF-κB Activator

Because TNFR2 signaling is transduced via activation of the NF-κB pathway, activators of NF-κB signaling can also be used in place of, or in combination with, administration of an enriched Treg composition for treating immunological disorders or conditions and infectious diseases according to the methods described above. For example, any one of the NF-κB activators described above can be used in the methods of treatment described above. The NF-κB activator can be used by itself or in combination with the composition enriched in Tregs.

### Methods for Producing an Enriched Lymphocyte Composition Depleted of Tregs

The invention also features methods for producing a composition enriched in lymphocytes (and depleted of Tregs) in vitro. Preferably the method produces a composition in which less than 10% of the cells (e.g., less than 10%, 5%, 3%, 1%, or 0.5%, or none of the cells) in the composition are Tregs. The method is similar to the method for producing a composition enriched in Tregs except a TNFR2 antagonist, such as an anti-TNFR2 monoclonal antibody, is used in place of a TNFR2 agonist. Additional TNFR2 antagonists that can be used in this method are described below.

The method generally involves the separation of T lymphocytes (e.g., CD4+ cells, CD25+ cells, or CD4+CD25+ cells) from human samples, e.g., human blood or bone marrow sample, followed by expansion of the cells during culturing by incubation of the cells with anti-CD3 and anti-CD28 antibodies. During the expansion step, the cells are contacted with a TNFR2 antagonist. The TNFR2 antagonist suppresses the proliferation of Tregs in the culture, thereby producing a composition that is enriched in lymphocytes and depleted of Tregs. Human-IL-2 and/or rapamycin may optionally be added to the cell culture during the expansion of cells.

After in vitro enrichment of lymphocytes (and depletion of Tregs), less than 10% (e.g., less than 10%, 9%, 8%, 7%, 5%, or 2% or substantially none) of the cells in this composition are Tregs. The method described above can result in approximately 2-fold (e.g., 2.5-fold, 3-fold, 3.5-fold, or 4-fold or more) enrichment of non-Treg lymphocytes (e.g., CD4+ T cells, CD8+ T cells, CD4+CD8+ T cells, B cells, natural killer cells, etc.). The enriched lymphocyte population may also include dendritic cells, monocytes, macrophages, and neutrophils.

### TNFR2 Antagonists

The TNFR2 antagonist that can be used in this method of the invention can include agents, such as an antibody, a peptide, a small molecule, and a protein that can bind to TNFR2 and suppress TNFR2 signaling. The TNFR2 antagonist can be an agent that, when contacted with CD4+ T cells, can stimulate the expression of cIAP but not the expression of TRAF2, TRAF3, or FOXP3.

The TNFR2 antagonist can be a monoclonal antibody that binds TNFR2. There are two epitopes of TNFR2 that the TNFR2 antagonist antibody can bind. The first epitope includes positions 48-67 (QTAQMCCSKCSPGQHAKVFC) of SEQ ID NO: 1 (amino acid sequence of human TNFR2). The second epitope includes position 135 (R) of SEQ ID NO: 1 (e.g., positions 135-153 (RLCAPLRKCRPGF) of SEQ ID NO: 1). For example, the TNFR2 antagonist antibody can be any one of Clone MAB726 (R&D Systems, Inc.) or Clone M1 (BD Biosciences). While each MAB726 and M1 binds the second epitope, an antibody of the invention may bind the first epitope or both epitopes. The TNFR2 antagonist antibody or antigen-binding fragment thereof can bind TNFR2 with a K_{D} of less than about 50 nM (e.g., less than about 30 nM, less than about 20 nM, less than about 10 nM, less than about 5 nM, less than about 2 nM, less than about 1 nM, less than about 900 pM, less than about 800 pM, or less than about 700 pM). The TNFR2 antagonist antibody or antigen-binding fragment thereof can bind TNFR2 with a Ko in the range of about 10 pM to about 50 nM (e.g., about 20 pM to about 30 nM, about 50 PM to about 20 nM, about 100 pM to about 5 nM, about 150 pM to about 1 nM, or about 200 pM to about 800 pM). The TNFR2 antagonist antibody avidity can be determined using methods known in the art (e.g., surface plasmon resonance. For example, MAB 726 binds TNFR2 with a K_{D} of 621 pM (determined by surface plasmon resonance (Pioneer SENSIQ®, Oklahoma City, OK)). The TNFR2 antagonist can also be a TNF-α mutein that is capable of binding to TNFR2 and suppressing downstream signaling.

The TNFR2 antagonist can function via downstream signaling by inhibition of the NF-κB pathway. Thus, the method of the invention can also include contacting the human sample e.g., a blood or bone marrow sample, with one or more inhibitors of the NF-κB pathway in order to achieve the same effect as that of using a TNFR2 antagonist. The NF-κB inhibitor can be a small molecule, a peptide, a protein, a virus, or a small non-coding RNA. In one embodiment, the NF-κB inhibitor that can be used in the methods to produce a composition enriched in lymphocytes can be any one or more of 2-(1,8-naphthyridin-2-yl)-Phenol, 5-Aminosalicylic acid, BAY 11-7082, BAY 11-7085, CAPE (Caffeic Acid Phenethylester), Diethylmaleate, Ethyl 3,4-Dihydroxycinnamate, Helenalin, Gliotoxin, NF-κB Activation Inhibitor II JSH-23, NFκB Activation Inhibitor III, Glucocorticoid Receptor Modulator, CpdA, PPM-18, Pyrrolidinedithiocarbamic acid ammonium salt, (R)-MG-132, Rocaglamide, Sodium Salicylate, QNZ, MG-132 [Z-Leu-Leu-Leu-CHO], Astaxanthin, (E)-2-Fluoro-4′-methoxystilbene, CHS-828, disulfiram, olmesartan, triptolide, withaferin, celastrol, tanshinone IIA, Ro 106-9920, cardamonin, BAY 11-7821, PSI, HU 211, ML130, PR 39, honokiol, CDI 2858522, andrographolide, and dithiocarbamates. The NF-κB inhibitor can also be a peptide inhibitor, e.g., a cell penetrating inhibitory peptide as described in May et al., Science, 2000 Sep. 1; 289(5484):1550-4 and in Orange and May, Cell Mol. Life Sci., 2008 November; 65(22):3564-91, each of which is incorporated herein by reference. Additional NF-κB inhibitors are also described in Gilmore and Herscovitch, Oncogene (2006) 25, 6887-6899; Nam, Mini Rev. Med. Chem., 2006 August; 6(8):945-51; and in U.S. Pat. No. 6,410,516, each of which is incorporated herein by reference.

Methods of Treatment Using a Treg-Depleted, Lymphocyte-Enriched Composition and/or Antagonist of the TNFR2 Signaling Pathway

The invention features methods for treating proliferative disorders, e.g., cancers, by administering a composition enriched in lymphocytes and depleted of Tregs to a patient in need thereof. The composition enriched in lymphocytes can be produced by the methods described above, for example, by contacting cells obtained from a human sample, e.g., blood or bone marrow sample, with a TNFR2 antagonist, e.g., a TNFR2 antagonist antibody, and/or an NF-κB inhibitor to produce a composition enriched in lymphocytes and depleted of Tregs. An NF-κB inhibitor may be used in methods of treating proliferative disorders, e.g., cancers, instead of the composition enriched in lymphocytes and depleted of Tregs or in combination with this composition. The invention also features methods for treating proliferative disorders, e.g., cancers, by administering a composition containing a TNFR2 antagonist (e.g., an anti-TNFR2 antagonist antibody) to a patient in need thereof.

The invention features methods for treating infectious diseases, by administering a composition enriched in lymphocytes and depleted of Tregs to a patient in need thereof. The composition enriched in lymphocytes can be produced by the methods described above, for example, by contacting cells obtained from a human sample, e.g., blood or bone marrow sample, with a TNFR2 antagonist, e.g., a TNFR2 antagonist antibody, and/or an NF-κB inhibitor to produce a composition enriched in lymphocytes and depleted of Tregs. An NF-κB inhibitor may be used in methods of treating infectious diseases, instead of the composition enriched in lymphocytes and depleted of Tregs or in combination with this composition. The invention also features methods for treating infectious diseases, by administering a composition containing a TNFR2 antagonist (e.g., an anti-TNFR2 antagonist antibody) alone to a patient in need thereof.

### Treatment of Proliferative Disorders Using an Enriched Lymphocyte Composition of the Invention

Non-Treg lymphocytes in a patient's blood can be expanded and administered back to the patient in order to treat a proliferative disorder (e.g., a cancer). The enriched lymphocyte composition can be administered alone or in combination with one or more anti-cancer agents known in the art.

The enriched lymphocyte composition can be prepared as follows: i) obtaining a sample e.g., a blood or bone marrow sample, from a human patient and isolating nucleated cells (e.g., lymphocytes, such as T lymphocytes, e.g., CD4+ cells, CD25+ cells, or CD4+CD25+ cells) present therein; ii) subjecting these cells to the lymphocyte expansion method described above to produce a composition enriched in lymphocytes and depleted of Tregs (e.g., a substantially homogenous population of lymphocytes in which Tregs comprise less than 10% of cells in the composition, preferably less than 5% of the cells in the composition or are absent in the expanded composition); and iii) introducing the composition enriched in lymphocytes into the patient without any post-expansion sorting of the lymphocytes. The above steps of the method of treatment can be performed in iterative cycles, where the number of cycles of treatment provided to a patient can be determined by the proliferative disorder being treated, the severity of the disorder, and/or the outcome of each treatment cycle, i.e., a change in the disease state. For example, changes in efficacy markers and/or in clinical outcomes can be used for determining the frequency with which blood or bone marrow should be drawn from a patient, the lymphocytes enriched (and Tregs depleted) from that blood, and the enriched lymphocytes administered to the patient. The enriched lymphocyte composition may also be prepared and stored for later use (e.g., frozen).

Preferably, the lymphocytes are obtained from a patient's own blood or bone marrow (i.e., autologous cells), although the following methods of treatment may also include the use of allogeneic lymphocytes with possible best fit HLA matching or from unrelated donors that have been expanded according to the above methods. Allogeneic lymphocytes preferentially share at least 4/6 HLA markers in common with the patient receiving the enriched lymphocyte composition.

Treatments for proliferative disorders according to the present invention may include inhibiting the NF-κB signaling pathway in combination with the administration of an enriched lymphocyte composition depleted of Tregs. Because TNFR2 signaling is transduced via the NF-κB pathway, the treatment of proliferative disorders according to the present invention can also include administering to a patient an inhibitor of the NF-κB pathway. For example, any one of the NF-κB inhibitors described above can be administered for treating a proliferative disorder. The NF-κB inhibitor can be administered by itself or in combination with the composition enriched in lymphocytes. The NF-κB inhibitor can also function independently of the TNFR2 signaling pathway.

Proliferative disorders that can be treated by administering the enriched lymphocyte/Treg depleted composition include one or more cancers selected from the group consisting of Acute Lymphoblastic Leukemia, Acute Lymphoblastic Leukemia, Acute Myeloid Leukemia, Adrenocortical Carcinoma; AIDS-Related Lymphoma, AIDS-Related Malignancies, Anal Cancer, Astrocytoma, Bile Duct Cancer, Bladder Cancer; Bone Cancer, Osteosarcoma/Malignant Fibrous Histiocytoma, Brain Stem Glioma, Visual Pathway and Hypothalamic Glioma, Breast Cancer, Bronchial Adenomas/Carcinoids, Chronic Lymphocytic Leukemia, Chronic Myelogenous Leukemia, Chronic Myeloproliferative Disorders, Clear Cell Sarcoma of Tendon Sheaths, Colon Cancer, Colorectal Cancer, Cutaneous T-Cell Lymphoma, Endometrial Cancer, Epithelial Cancer, Esophageal Cancer, Ewing's Family of Tumors, Extracranial Germ Cell Tumor, Extragonadal Germ Cell Tumor, Extrahepatic Bile Duct Cancer, Eye Cancer, Intraocular Melanoma, Retinoblastoma, Gallbladder Cancer, Gastric (Stomach) Cancer, Hairy Cell Leukemia, Head and Neck Cancer, Hepatocellular (Liver) Cancer, Hodgkin's Lymphoma, Hypopharyngeal Cancer, Kaposi's Sarcoma, Kidney Cancer, Laryngeal Cancer, Pituitary Cancer, Plasma Cell Neoplasm/Multiple Myeloma, Pleuropulmonary Blastoma, Skin Cancer, Small Cell Lung Cancer, Small Intestine Cancer, Soft Tissue Sarcoma, Squamous Neck Cancer, Testicular Cancer, Thyroid Cancer, Urethral Cancer, Uterine Sarcoma, and Vaginal Cancer. The proliferative disorders can also include solid tumors including malignancies (e.g., sarcomas, adenocarcinomas, and carcinomas) of the various organ systems, such as those of brain, lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine.

Administration and dosage of the enriched lymphocyte composition in the method for treating a proliferative disease are discussed herein below.

### Treatment of an Infectious Disease Using an Enriched Lymphocyte Composition of the Invention

The invention also features methods of treating infectious diseases caused by any one or more of a virus, bacteria, a fungus, or a parasite. The methods involve administering a composition enriched in lymphocytes (e.g., CD8+ T cells, B cells, or natural killer cells) and depleted of Tregs. This composition can be produced by the methods described above. The methods of the invention can be used for treating viral infections caused by, e.g., a member of the Flaviviridae family (e.g., a member of the Flavivirus, Pestivirus, and Hepacivirus genera), which includes the hepatitis C virus, Yellow fever virus; Tick-borne viruses, such as the Gadgets Gully virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus and the Negishi virus; seabird tick-borne viruses, such as the Meaban virus, Saumarez Reef virus, and the Tyuleniy virus; mosquito-borne viruses, such as the Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus; and viruses with no known arthropod vector, such as the Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, and the Cell fusing agent virus; a member of the Arenaviridae family, which includes the Ippy virus, Lassa virus (e.g., the Josiah, LP, or GA391 strain), lymphocytic choriomeningitis virus (LCMV), Mobala virus, Mopeia virus, Amapari virus, Flexal virus, Guanarito virus, Junin virus, Latino virus, Machupo virus, Oliveros virus, Paraná virus, Pichinde virus, Pirital virus, Sabiá virus, Tacaribe virus, Tamiami virus, Whitewater Arroyo virus, Chapare virus, and Lujo virus; a member of the Bunyaviridae family (e.g., a member of the Hantavirus, Nairovirus, Orthobunyavirus, and Phlebovirus genera), which includes the Hantaan virus, Sin Nombre virus, Dugbe virus, Bunyamwera virus, Rift Valley fever virus, La Crosse virus, California encephalitis virus, and Crimean-Congo hemorrhagic fever (CCHF) virus; a member of the Filoviridae family, which includes the Ebola virus (e.g., the Zaire, Sudan, Ivory Coast, Reston, and Uganda strains) and the Marburg virus (e.g., the Angola, Ci67, Musoke, Popp, Ravn and Lake Victoria strains); a member of the Togaviridae family (e.g., a member of the Alphavirus genus), which includes the Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O'nyong'nyong virus, and the chikungunya virus; a member of the Poxviridae family (e.g., a member of the Orthopoxvirus genus), which includes the smallpox virus, monkeypox virus, and vaccinia virus; a member of the Herpesviridae family, which includes the herpes simplex virus (HSV; types 1, 2, and 6), human herpes virus (e.g., types 7 and 8), cytomegalovirus (CMV), Epstein-Barr virus (EBV), Varicella-Zoster virus, and Kaposi's sarcoma associated-herpesvirus (KSHV); a member of the Orthomyxoviridae family, which includes the influenza virus (A, B, and C), such as the H5N1 avian influenza virus or H1N1 swine flu; a member of the Coronaviridae family, which includes the severe acute respiratory syndrome (SARS) virus; a member of the Rhabdoviridae family, which includes the rabies virus and vesicular stomatitis virus (VSV); a member of the Paramyxoviridae family, which includes the human respiratory syncytial virus (RSV), Newcastle disease virus, hendravirus, nipahvirus, measles virus, rinderpest virus, canine distemper virus, Sendai virus, human parainfluenza virus (e.g., 1, 2, 3, and 4), rhinovirus, and mumps virus; a member of the Picornaviridae family, which includes the poliovirus, human enterovirus (A, B, C, and D), hepatitis A virus, and the coxsackievirus; a member of the Hepadnaviridae family, which includes the hepatitis B virus; a member of the Papillamoviridae family, which includes the human papilloma virus; a member of the Parvoviridae family, which includes the adeno-associated virus; a member of the Astroviridae family, which includes the astrovirus; a member of the Polyomaviridae family, which includes the JC virus, BK virus, and SV40 virus; a member of the Calciviridae family, which includes the Norwalk virus; a member of the Reoviridae family, which includes the rotavirus; and a member of the Retroviridae family, which includes the human immunodeficiency virus (HIV; e.g., types 1 and 2), and human T-lymphotropic virus Types I and II (HTLV-1 and HTLV-2, respectively)).

The methods of the invention can also be used for treating bacterial infections. Examples of bacterial infections that may be treated include, but are not limited to, those caused by bacteria within the genera Salmonella, Streptococcus, Bacillus, Listeria, Corynebacterium, Nocardia, Neisseria, Actinobacter, Moraxella, Enterobacteriacece, Pseudomonas, Escherichia, Klebsiella, Serratia, Enterobacter, Proteus, Salmonella, Shigella, Yersinia, Haemophilus, Bordatella, Legionella, Pasteurella, Francisella, Brucella, Bartonella, Clostridium, Vibrio, Campylobacter, and Staphylococcus.

The methods of the invention can also be used for treating parasitic infections caused by a protozoan parasite (e.g., an intestinal protozoa, a tissue protozoa, or a blood protozoa) or a helminthic parasite (e.g., a nematode, a helminth, an adenophorea, a secementea, a trematode, a fluke (blood flukes, liver flukes, intestinal flukes, and lung flukes), or a cestode). Exemplary protozoan parasites include Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Toxoplasma gondii, Plasmodium vivax, Plasmodium ovale, Plasmodium malariae, Plasmodium falciparum, Trichomonas vaginalis, and Histomonas meleagridis. Exemplary helminthic parasites include richuris trichiura, Ascaris lumbricoides, Enterobius vermicularis, Ancylostoma duodenale, Necator americanus, Strongyloides stercoralis, Wuchereria bancrofti, and Dracunculus medinensis, Schistosoma mansoni, Schistosoma haematobium, Schistosoma japonicum, Fasciola hepatica, Fasciola gigantica, Heterophyes heterophyes, and Paragonimus westermani, Taenia solium, Taenia saginata, Hymenolepis nana, and Echinococcus granulosus.

The methods of the invention can also be used for treating fungal infections. Examples of fungal infections that may be treated include, but are not limited to, those caused by, e.g., Aspergillus, Candida, Malassezia, Trichosporon, Fusarium, Acremonium, Rhizopus, Mucor, Pneumocystis, and Absidia.

Administration and dosage of the lymphocyte-enriched composition in methods for treating an infectious disease are discussed herein below.

### Dosage

The composition enriched in Tregs can be administered to a patient in need thereof one or more times per day, week, month (e.g., one or more times every 2 weeks), or year depending on the severity of the disease and change in disease state of the patient during the treatment. Generally it is expected that a typical dosage would include 5×10⁵ to 5×10¹² (e.g., 5×10⁵, 5×10⁶, 5×10⁷, 5×10⁸, 5×10⁹, 5×10¹⁰, 5×10¹¹, or 5×10¹²) Tregs. Disease metrics, such as severity of symptoms, change in symptoms, patient response to treatment, any adverse effects of treatment, and/or effect of any additional treatment(s), can be used to determine the frequency of treatment and the dosage, i.e., the number of Tregs to be administered to a patient.

The composition enriched in lymphocytes (and depleted of Tregs) can be administered one or more times per day, week, month (e.g., one or more times every 2 weeks), or year depending on the severity of the disease and change in disease state of the patient during the treatment. Preferably, less than 10% of the cells (e.g., less than 9%, 8%, 7%, 5%, %, 1% or none of the cells) in this composition enriched in lymphocytes are Tregs. Generally it is expected that a typical dosage would include 5×10⁵ to 5×10¹² (e.g., 5×10⁵, 5×10⁶, 5×10⁷, 5×10⁸, 5×10⁹, 5×10¹⁰, 5×10¹¹, or 5×10¹²) cells in the enriched lymphocyte composition. Disease metrics, such as severity of symptoms, change in symptoms, patient response to treatment, any adverse effects of treatment, and/or effect of any additional treatment(s), can be used to determine the frequency of treatment and the dosage, i.e., the number of lymphocytes to be administered to a patient.

### Administration

Generally, the compositions of the invention (e.g., the composition enriched in Tregs or the composition enriched in lymphocytes and depleted of Tregs) can be administered in any medically useful form. For example, such compositions may include the addition of compounds, e.g., adjuvants, preservatives, carriers, excipients, diluents, anti-bacterial or anti-mycotic agents, anti-inflammatory agents, and/or anti-cancer agents, where appropriate. The compositions of the invention can be administered intravenously, intramuscularly, orally, by inhalation, parenterally, intraperitoneally, intraarterially, transdermally, sublingually, nasally, transbuccally, liposomally, adiposally, ophthalmically, intraocularly, subcutaneously, intrathecally, orally, or locally, and they are formulated, as appropriate, depending on the chosen route of administration.

### Administration of Antibodies of the Invention

Pharmaceutical compositions containing an anti-TNFR2 antibody of the invention (e.g., an anti-TNFR2 antagonist antibody) are prepared for storage by mixing the antibody having the desired degree of purity with optional physiologically acceptable carriers, excipients or stabilizers in the form of aqueous solutions, lyophilized or other dried formulations. The acceptable excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21^{st} edition, Ed. Gennaro; Lippincott, Williams & Wilkins (2005), a well-known reference text in this field, and in the USP/NF (United States Pharmacopeia and the National Formulary). Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Other exemplary excipients are described in Handbook of Pharmaceutical Excipients, 6^{th} Edition, Rowe et al., Eds., Pharmaceutical Press (2009).

The compositions of the invention may be prepared in a pharmaceutically acceptable carrier or excipient. Such suitable carriers or excipients may be selected from, for example, water, saline (e.g., phosphate-buffered saline (PBS) or acetate-buffered saline (ABS), or Ringer's solution), dextrose, glycerol, ethanol, or the like and combinations thereof. In addition, if desired, a composition for administration to a mammal can contain minor amounts of auxiliary substances, such as wetting or emulsifying agents, or pH buffering agents that enhance the effectiveness of the composition. The compositions of the invention may also be prepared in any acceptable salt formulation. Other agents that may be used in preparation of the compositions of the invention include, e.g., adjuvants, preservatives, diluents, anti-bacterial or anti-mycotic agents, anti-inflammatory agents, and/or anti-cancer agents, where appropriate.

The compositions may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington: The Science and Practice of Pharmacy, 21^{st} edition, Ed. Gennaro; Lippincott, Williams & Wilkins (2005).

The compositions to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the immunoglobulin of the invention, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The compositions containing one or more anti-TNFR2 antibodies (e.g., anti-TNFR2 antagonist antibody) may be administered to a patient prior to the development of symptoms of a proliferative disease or an infectious disease or the compositions may be administered to the patient after diagnosis with a proliferative disease or an infectious disease after presentation with one or more (e.g., 1, 2, 3, 4, or 5) symptoms of the disease. The dosage of the anti-TNFR2 antibodies depends on the patient's state of health, but generally ranges from about 0.1 mg to about 400 mg of an antibody per dose (e.g., 1 mg, 5 mg, 10 mg, 20 mg, 50 mg, 100 mg, 200 mg, 300 mg, or 400 mg or more per dose).

The compositions may be administered to a patient in one or more doses (e.g., 2, 3, 4, 5, 6, 7, 8, 9, or 10 or more doses). If more than one dose is administered, the doses may be administered via the same mode of administration (e.g., intravenous administration) or by different modes of administration (e.g., intravenous and intramuscular administration). The patient may also be administered different doses at different times. For example, the patient may be administered a higher initial dose and lower subsequent doses over the course of treatment or vice versa.

The compositions may be administered daily, weekly, monthly, or yearly. For example, a dose of the composition may be administered twice daily, biweekly, bi-annually, tri-annually, or quarterly. The dose of the composition may be determined by a skilled physician upon consideration of a subject's clinical symptoms and/or physical condition (e.g., weight, sex, height, and severity of the proliferative or infectious disease). The composition may be administered by intravenous, intradermal, parenteral, intra-arterial, subcutaneous, intramuscular, intraorbital, topical, intraventricular, intraspinal, intraperitoneal, intranasal, intracranial, or oral administration.

### Kits of the Invention

The invention features a kit for the production of a composition enriched in Tregs. The kit can include a TNFR2 agonist (e.g., a TNFR2 agonist antibody) or an NF-κB activator (e.g., one or more of the NF-κB activators described above), reagents and/or devices for isolating a human sample, e.g., a blood or bone marrow sample, reagents and/or devices for isolating blood or bone marrow cells (e.g., CD4+CD25+ cells) from the sample, and reagents for culturing the blood or bone marrow cells (e.g., anti-CD3 antibody, anti-CD28 antibody, interleukin-2, and/or rapamycin). Additionally the kit of the invention can also include instructions for performing the method of the invention, e.g., instructions for isolating a blood or bone marrow sample and blood or bone marrow cells therefrom, instructions for contacting blood or bone marrow cells with a TNFR2 agonist, and/or instructions for culturing, harvesting and/or storing the enriched Tregs. The kit of the invention can also include reagents and instructions for assaying the expression of various marker genes that can be used to characterize the Tregs. For example, these can include reagents and instructions for detecting the mRNA or protein levels of one or more of FOXP3, CTLA4, TNFR2, CD62L, Fas, HLA-DR, CD45RO, CD127, CCR5, CCR6, CCR7, CXCR3, IFN-gamma, IL10, and ICOS.

The invention features a kit for the production of a composition enriched in lymphocytes and depleted of Tregs. The kit can include a TNFR2 antagonist (e.g., a TNFR2 antagonist antibody) or an NF-κB inhibitor (e.g., one or more of the NF-κB inhibitors described above), reagents and/or devices for isolating a human blood or bone marrow sample, reagents and/or devices for isolating blood or bone marrow cells (e.g., T lymphocytes, such as CD4+ cells, CD25+ cells, or CD4+CD25+ cells) from the sample, and reagents for culturing the blood cells (e.g., anti-CD3 antibody, anti-CD28 antibody, interleukin-2, and/or rapamycin). Additionally the kit of the invention can also include instructions for performing the method of the invention, e.g., instructions for isolating a blood or bone marrow sample and blood or bone marrow cells therefrom, instructions for contacting blood cells with a TNFR2 antagonist, and/or instructions for culturing, harvesting and/or storing the enriched lymphocytes. The kit of the invention can also include reagents and instructions for assaying the expression of various marker genes that can be used to characterize the lymphocytes. For example, these can include reagents and instructions for detecting the mRNA or protein levels of one or more of FOXP3, TRAF2, TRAF3, and cIAP.

The invention also features kits that include a composition containing an anti-TNFR2 antibody (e.g., an anti-TNFR2 antagonist antibody), a pharmaceutically-acceptable carrier or excipient, and, optionally, other agents as described herein; the composition contains an effective amount of the anti-TNFR2 antibody for treating a proliferative disease or an infectious disease. The kits may include instructions explaining how a practitioner (e.g., a physician, nurse, or patient) may administer the composition contained therein. Furthermore, the kits may also include additional components, such as one or more additional components described above, instructions or administration schedules for a patient suffering from a proliferative disease or an infectious disease, and, optionally, a device(s) for administering the composition (e.g., a syringe).

The following examples are meant to illustrate the invention. They are not meant to limit the invention in any way.

### EXAMPLES

### Materials and Methods

Human Subjects and TNF-α Induction with BCG Vaccine

Two BCG vaccinations were used to induce TNF-α. Administration of BCG was approved by the Human Studies Committee at Massachusetts General Hospital and by the FDA (NCT00607230).

For the double-blinded placebo-controlled trial, one subject was injected with BCG at a dose of 1.6-3.2×10⁶ cfu and the placebo subject was injected with saline. The BCG or saline injection was administered intradermally on two occasions four-weeks apart. All blood samples were blinded and simultaneously sent to the laboratory for monitoring TNF-α and Treg levels.

### Reagents and Flow Cytometry

Recombinant human TNF-α was purchased from Leinco Technologies (St. Louis, MO), and recombinant human IL-2 was purchased from Sigma-Aldrich (St. Louis, MO). Monoclonal antibodies against TNFR1 and TNFR2 used for screening purposes were from internal sources and external vendors (Table 1). External vendors included R&D Systems, Inc., Hycult-Biotechnology, BD-Pharmingen, Accurate, Abcam and Sigma. All other antibodies were purchased from BD-Biosciences. Intracellular staining of FOXP3 and CD152 were performed using either FOXP3 Fix/Perm Buffer set (Biolegend) or Human FOXP3 Buffer set (BD Biosciences). The avidities of MAB726 and M1 antibodies for TNFR2 were determined using surface plasmon resonance on Pioneer SENSIQ® (SensiQ Technologies, Oklahoma City, OK).

TABLE 1

| Clone | MR2-1 | 80M2 | MAB726 | M1 | MAB2261 |
|---|---|---|---|---|---|
| Isotype | Mouse | Mouse | Mouse | Rat | Mouse |
| | IgG1 | IgG1 | IgG1 | IgG2b | IgG2A |
| Properties | Agonist | Neutral | Antagonist | Antagonist | Agonist |
| Vendor | Cell | Cell | R&D | BD | R&D |
| | Sciences | Sciences | | Biosciences | |

CD4+ Cell Isolation, Induction of FOXP3, and Expansion of CD4⁺CD25⁺ Cells

CD4+ T cells were isolated using Dynal CD4 Positive Isolation Kit (Invitrogen). Extraction of CD25 positive cells was subsequently performed after CD4+ isolation using DYNABEADS® CD25 and DETACHABEAD™ CD4/CD8 (Invitrogen). After isolation, 2×10⁴ cells were cultured in 96-round-bottom well plate. DYNABEADS® for human Treg Expander (Invitrogen) (DYNABEADS® coupled with anti-CD3 and anti-CD28 monoclonal antibodies) was added at a beads-to-cell ratio of 2:1. In selected wells, TNF-α (20 ng/ml), TNFR2 mAbs (2.5 μg/ml), rapamycin (1 μM, EMD Biosciences, San Diego, CA) were added. After two days, IL-2 (200 U/ml) was added to the culture. Half of the media was changed every 2 to 3 days containing rapamycin (until day 7) and 100 U/ml of IL-2. On day 9, additional TNF-α or TNFR2 mAbs were supplied into the media. On day 16, cells were harvested, DYNABEADS® Human Treg Expander was removed, washed and rested. On the following day, cells were analyzed.

### Intracellular Staining

Expanded CD4⁺CD25⁺ cells were stimulated with phorbol myristate acetate (PMA) (2 ng/ml) and ionomycin (500 ng/ml) (Sigma) for 24 hours. Monensin (GOLGISTOP™, BD Biosciences) was added for the last 4 hours of incubation. Cells were fixed and permeabilized using Human FOXP3 Buffer Set, followed by staining with fluorochrome-conjugated IFNγ and IL-10 mAbs.

mRNA Isolation

Isolated CD4+ cells were incubated in presence of IL-2 (50 U/ml) with or without TNFR2 mAbs (2.5 μg/ml). After 3 hours, cells were collected and total RNA was isolated using RNAQUEOUS™-4PCR kit (Ambion, Austin, TX). The extracted RNA was reverse transcribed using High Capacity cDNA Reverse Transcription Kit (APPLIED BIOSYSTEMS™, Foster City, CA).

### Cell Proliferation and Suppression Assays

For CD4 proliferation experiments, CD4+ cells were stained with 1 μM carboxyfluorescein diacetate succinimidyl ester (CFSE). Cells were plated at the density of 2×10⁵ cells/well in 96-well plate with anti-CD3mAbs. Four days later, cells were collected and analyzed.

For Treg suppression assay, autologous peripheral blood mononuclear cells (PBMCs) were used as responders. PBMC were collected using FICOLL-PAQUE™, cryopreserved at −80° C., and thawed the day before mixing with Tregs and rested overnight in RPMI 1640 and 10 U/ml IL-2. The next day, responder cells were stained with CFSE (1 μM). Responder cells (5×10⁴ cells) and expanded Tregs were mixed at various ratios, and stimulated with anti-CD3mAb and IL-2. After 4 days, cells were collected and analyzed.

### Statistical Analysis

All data analyses were performed by the paired Student t test using GraphPad PRISM®-5 software (GraphPad Software, La Jolla, CA). I considered two-sided p value 0.05 as significant.

### Example 1: Clinical Trial Induction of Human Tregs

Unexpanded, naturally occurring human Tregs are heterogeneous and rare in blood. Homogeneous populations of Tregs are difficult to expand in vitro even with multiple ligand mixtures. With the goal of expanding sufficient numbers of homogeneous populations of human Treg cells TNF-α, I first sought to confirm or refute an increase in Treg concentrations by induction with native TNF-α. Because an FDA-approved version of TNF-α does not exist and manufacturing of a stable form is difficult, I administered a well-known, strong inducer of TNF-α, Bacillus Calmette Guérin (BCG), a generic vaccine already on the market for decades for tuberculosis and bladder cancer. This method of inducing endogenous TNF-α eliminated the manufacturing problems of TNF-α that forms unnatural monomers, dimers and trimers with differential cellular effects.

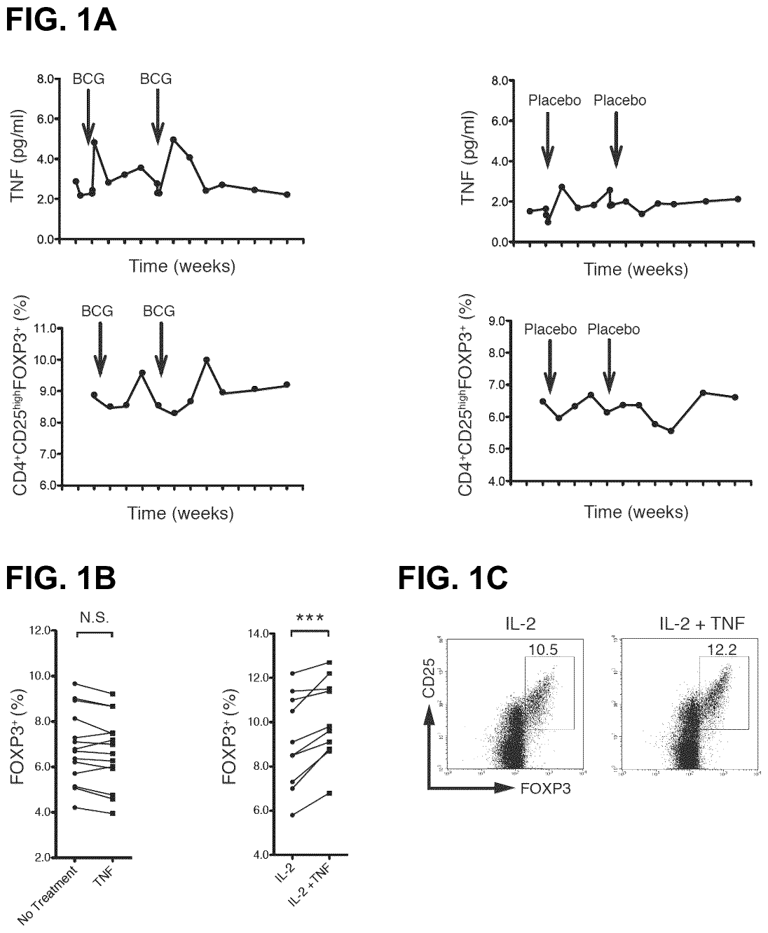

The small, double-blinded placebo-controlled clinical trial enrolled two subjects. One human subject received BCG injections (1.6-3.2×10⁶ cfu/injection) and the placebo received saline, twice, 4-weeks apart. Both were monitored weekly for 20-weeks to study the pharmacokinetics of TNF-α and Treg induction. After each injection TNF-α induced Tregs in a bi-modal fashion with slightly delayed kinetics (FIG. 1A, left panels). After 20 weeks of observation, saline injections induced neither TNF-α nor Tregs. The total CD4+ cell counts did not change in BCG and placebo patients other than in the percentages of CD4+CD25^{hi}FOXP3+ depicted in FIG. 1A. This in vivo evidence confirms endogenous TNF-α in vivo increases the numbers of Tregs but since TNF-α binds to both the TNFR1 and TNFR2 receptors, it does not clarify which receptor was more central for the Tregs effect.

### Example 2: Functional Effects of TNFR Monoclonal Antibodies and Signaling Pathways

I cultured freshly isolated human CD4+ cells from 14 human subjects only with TNF-α for 16 hours (FIG. 1B). While finding no induction of Tregs, assessed by inducible FOXP3, I observed a significant increase in Tregs after adding IL-2. Co-incubation of TNF-α and IL-2 produced a significant increase in Tregs over IL-2 alone (FIG. 1B). IL-2 is important for Tregs induction and maintenance in mice. Findings were confirmed by flow cytometry, in which co-incubation increased the percentage of CD4+CD25^{hi} FOXP3 cells in human cultured cells from blood (FIG. 1C). Thus, the data in FIGS. 1B and 1C confirm that TNF-α can induce a homogeneous population of Tregs in vitro.

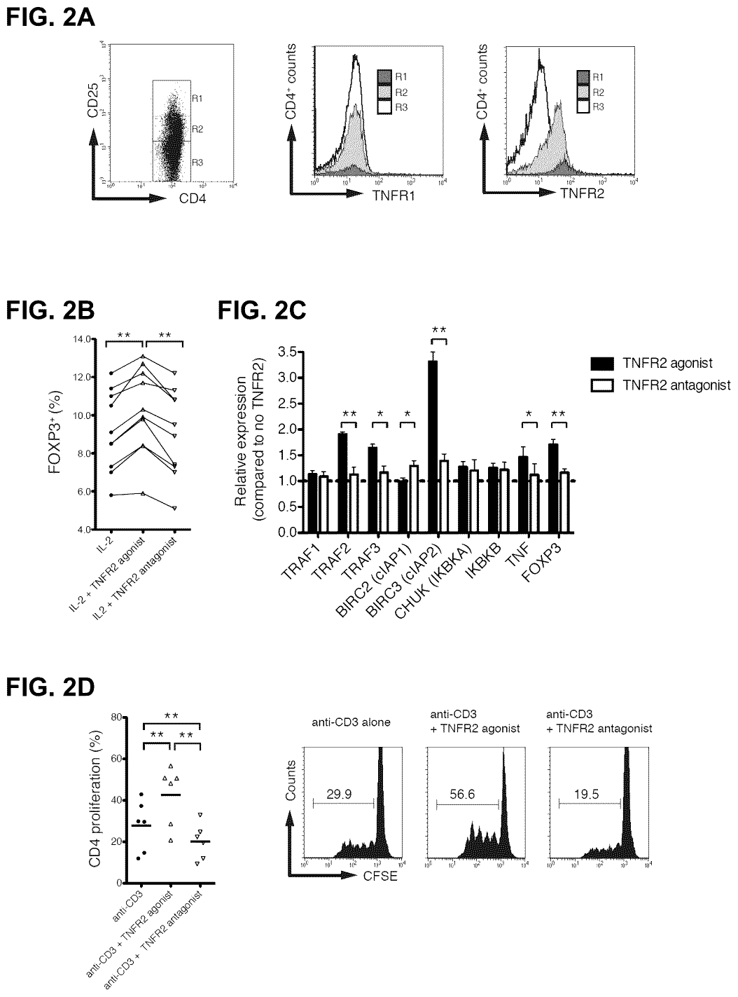

In freshly isolated CD4+ cells, I examined expression levels of each TNFR in relation to CD25+ expression. TNFR1 expression on CD4+ cells, regardless of CD25+ expression levels, was unchanged using flow cytometry (FIG. 2A, middle panel), whereas TNFR2 preferentially expressed CD4+CD25^{hi} Tregs by nearly a factor of 10 (FIG. 2A, right panel). This confirms earlier studies that TNFR2 is more densely expressed on Tregs.

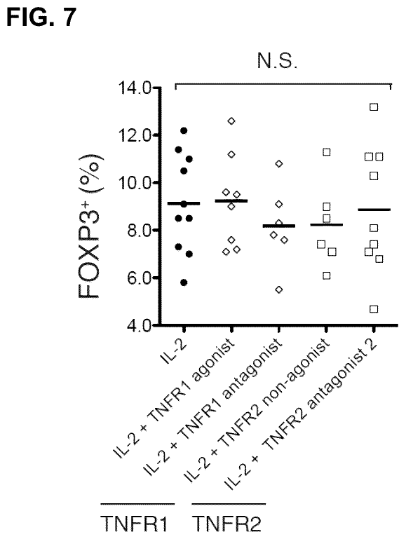

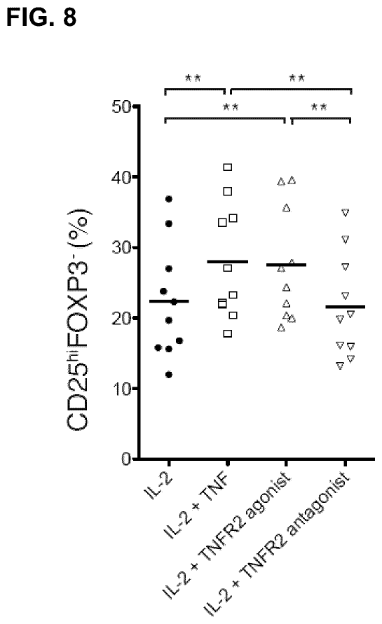

Screening several TNFR1 and TNFR2 monoclonal antibodies (mAbs) on isolated CD4+ cells sampled from fresh human blood enabled selective study of each TNF receptor, unlike studying TNF-α, which acts through both receptors and can have manufacturing problems from the use of E. coli and yeast systems. Although most of the screened TNFR1 or TNFR2 mAbs failed to induce or suppress FOXP3+ Tregs after stimulation by presence of IL-2 for 16 hours (FIGS. 7 and 8), I found two types of TNFR2 mAbs with significant, and opposing, effects on FOXP3 induction (FIG. 2B). Studying freshly cultured cells from 10 subjects, one TNFR2 antibody significantly induced FOXP3 expression in CD4+ human T cells (which I designated the “TNFR2 agonist”), whereas the other TNFR2 monoclonal suppressed intracellular FOXP3 expression (which I designated the “TNFR2 antagonist”) (FIG. 2B). Thus, I have identified M1 and MAB726 as TNFR2 antagonists.

Having identified two functionally-opposing types of TNFR2 mAbs, I measured their effects on isolated CD4+ T cells by examining downstream mRNA expression in signaling proteins specific to TNFR2 activation. After 24 h stimulation by the TNFR2 agonist or antagonist, relative mRNA expression was significantly different. The TNFR2 agonist stimulated expression of TNF, TRAF2, TRAF3, cIAP2 and FOXP3. In contrast, the TNFR2 antagonist stimulated expression of cIAP1, but not TRAF2, TRAF3 or FOXP3 (FIG. 2C).

The effects of the TNFR2 agonist and antagonist were studied on purified human CD4+ T cells co-cultured with anti-CD3 or IL-2. When CD4+ proliferation was studied with anti-CD3 combined with the TNFR2 agonist, the highest degree of proliferation was shown by the agonist. In contrast, the TNFR2 antagonist (e.g., M1 or MAB726) suppressed CD4+ proliferation even relative to the control, anti-CD3 alone (FIG. 2D, left-most panel). The same findings were observed after 4 days by directly measuring CD4+ proliferation by flow cytometry and measuring CD4+ proliferation by CFSE dilution (FIG. 2D, three right-most panels). Thus, the opposing effects of TNFR2 agonist treatment versus TNFR2 antagonist treatment on Tregs have been demonstrated, as shown in FIGS. 2B-2D.

Despite high expression of TNFR2 on Tregs, some TNFR2 expression is also observable on CD4+ T cells that are not true Tregs because they only express intermediate levels of CD25, i.e., CD4+CD25^{mid} cells. I therefore studied the impact of overnight incubation on CD25^{mid} cell subpopulations of IL-2 alone, IL-2 and TNF-α, or IL-2 and TNFR2 agonist, or IL-2 and TNFR2 antagonist. I found a rise in the proportion of CD25^{hi}FOXP3− cells similar to effector cells with IL-2 and TNF-α stimulation alone, or IL-2 and TNFR2 agonist alone (FIG. 8). However, I observed suppression with IL-2 and TNFR2 antagonist relative to the other three groups. Therefore, the TNFR2 agonist and antagonist, studied by the same assay, showed opposing trends on the same CD25+ FOXP3− cell population. Thus, the addition of a TNFR2 antagonist (e.g., M1 or MAB726) inhibited expression of Foxp3 on CD4+CD25+ T cells.

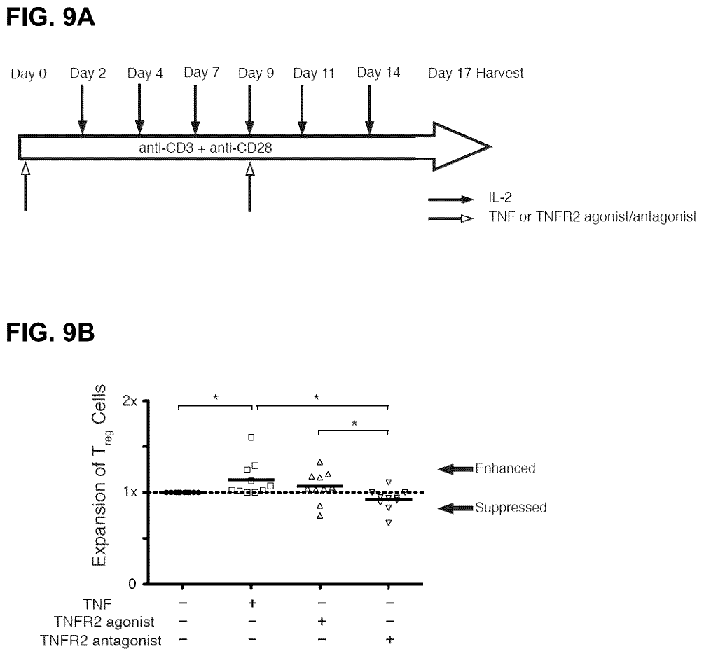

I separated fresh human blood to obtain pure CD4+ and CD25^{hi} co-expressing Tregs (FIGS. 3A-3C). I purified and expanded these Tregs in vitro using standard protocols of anti-CD3 anti-CD28 plus IL-2 for 16 days (FIG. 3A), then rested them overnight before counting. I added rapamycin (until day 7) because it selectively expands the highest number of Tregs with greatest capacity for suppressing CD8+ cells. This process successfully produced CD4+CD25^{hi} Tregs (FIG. 3B). I assessed Treg expansion by treatment group: no treatment, treatment with TNF-α, TNFR2 agonist, or TNFR2 antagonist. The TNFR2 agonist outperformed every other group, expanding Tregs at least twofold higher than no treatment or antagonist treatment. The latter suppressed expansion because its effect was less than that of no treatment. Because rapamycin is known to inhibit proliferation, I examined the effects of treatment without rapamycin, yet found similarly opposing effects between agonist versus antagonist treatment, albeit at smaller mean absolute values (FIG. 9B). The yields of expanded cells tended to be less without rapamycin, but the agonist still expanded Tregs.

### Example 3: TNFR2 Agonist Expansion and Homogeneity of Tregs

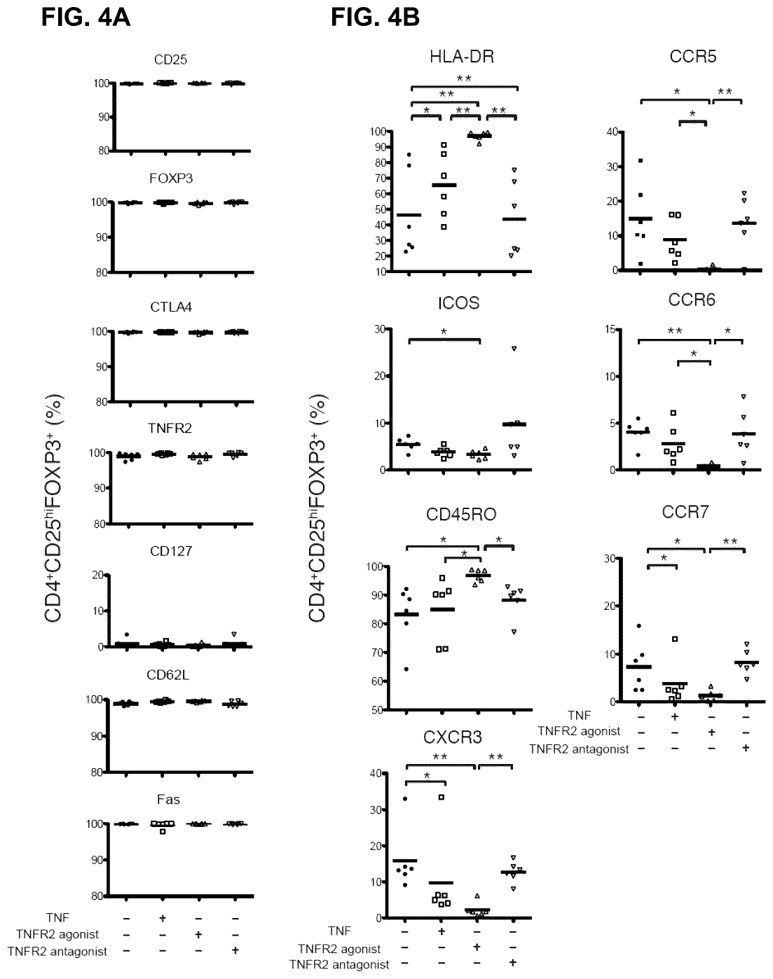

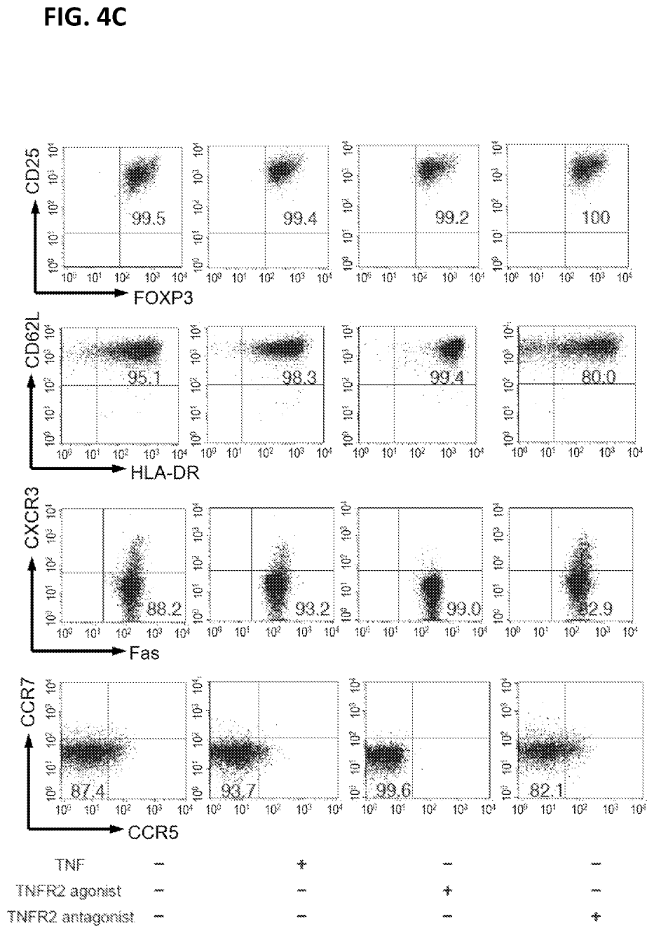

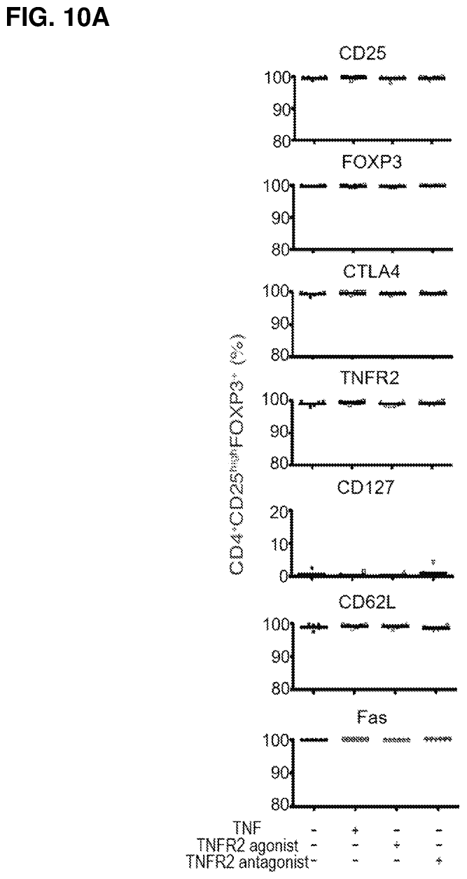

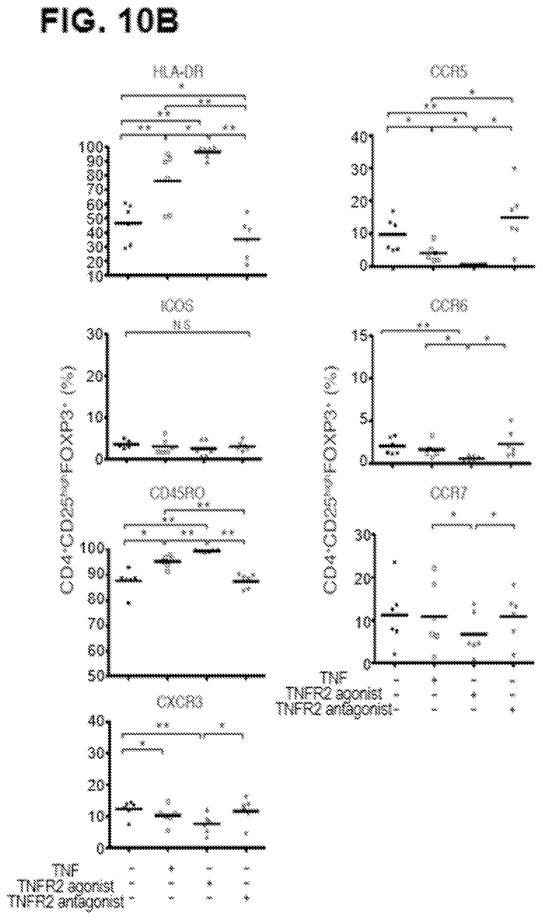

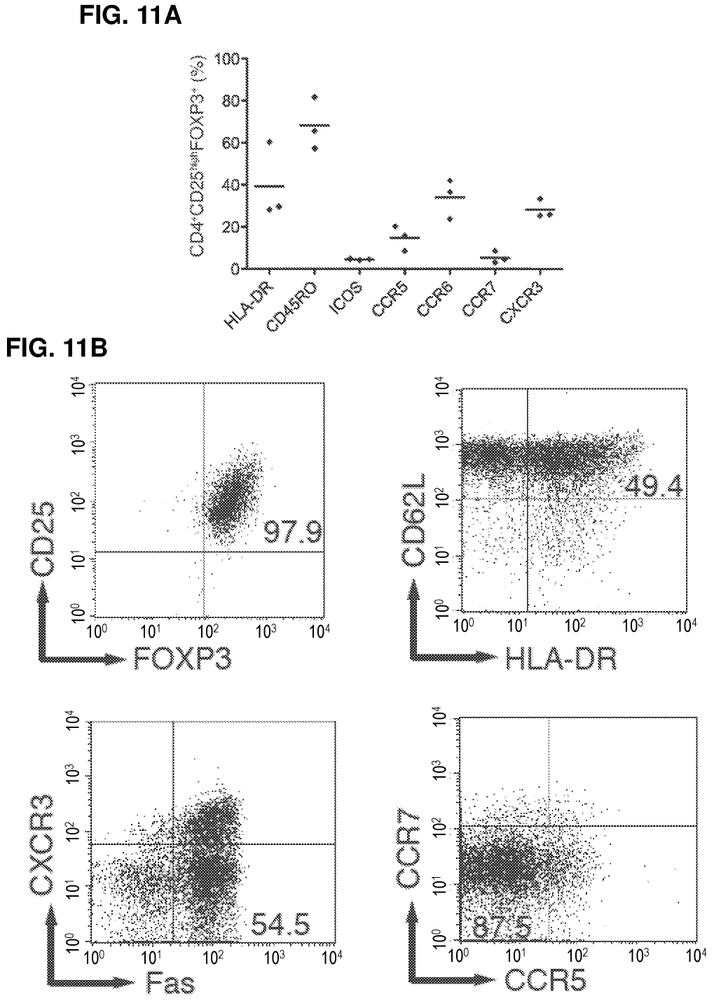

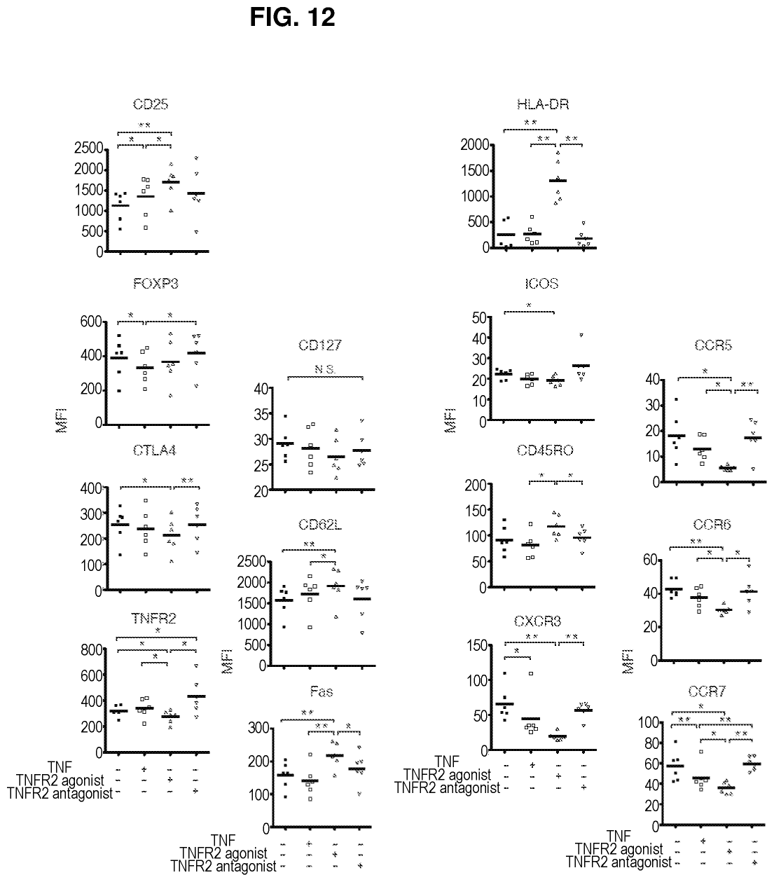

I next investigated whether in vitro Tregs, treated by TNFR2 agonist, possessed more homogeneous Treg cell surface markers than those treated by the antagonist. Comparing phenotypes for 14 cell surface markers, all treatment groups highly expressed Treg signature markers FOXP3 and CD25 (FIG. 4A). The expression levels of FOXP3 were similar to levels before treatment. However, CD25+ expression was much higher after agonist treatment, which can be considered an expansion effect rather than an antagonist effect (data not shown). Nearly 100% of expanded CD25^{hi} Tregs in each group were positive for CTLA4, TNFR2, CD62L, Fas, and negative for CD127 (FIG. 4A). Tregs treated with TNFR2 antagonist also maintained expression for these markers. In contrast, several other surface markers, such as HLA-DR, ICOS, CD45RO and chemokine receptors, were differentially expressed between the agonist vs. antagonist treatment (FIGS. 4B and 4C and FIGS. 10A and 10B). Similar results were observed in Tregs expanded without rapamycin (FIGS. 10A and 10B). Tregs expanded by TNFR2 agonist-relative to most other comparator groups, especially the TNFR2 antagonist-yielded a surprisingly homogeneous population of cells with this phenotype: CD4⁺CD25^{hi}FOXP3⁺CTLA4⁺TNFR2⁺CD127⁻ CD62L⁺Fas⁺HLA⁻DR⁺CD45RO⁺CCR5⁻CCR6⁻CCR7⁻CXCR3⁻ICOS⁻. The mean fluorescence intensity (MFI), a direct measure of the average density of the protein per cell, similarly revealed that for most surface markers, the TNFR2-agonist treated cells showed opposing expression levels compared to TNFR2-antagonist treated cells (FIG. 12). Further investigation should define whether these expanded cells maintain phenotypic homogeneity over time but the evidence from this conventional expansion protocol shows that they were more homogeneous than other groups. Before treatment, Treg markers were more heterogeneous (FIGS. 11A and 11B). Unexpanded, naturally occurring human Tregs are heterogeneous populations. In vitro studies of mixed Treg populations, which include CD45RO⁺FOXP3^{low} T cells, produce pro-inflammatory cytokines. This particular phenotype is found in up to 50% of FOXP3+ T cells.

One of the most upregulated markers by TNFR2 agonist-treated Tregs was HLA-DR, which is reported to have higher suppressive activity against CD8 T+ cells, suggestive of an effector Treg. In contrast to HLA-DR, all four chemokine receptors were strongly down-regulated. Although the lack of chemokine receptors might result in failure to migrate to the site of inflammation, another homing receptor, CD62L, which was highly expressed in all treatment groups (FIGS. 4A-4C), is crucial for entering the site of pathogenic T cell presence in acute GVHD. The fact that agonist-treated Tregs were CD45RO+ and CCR7− and displayed significantly higher expression levels of Fas, measured by MFI (FIG. 12), contributes to the view that they are activated effector Tregs.

### Example 4: TNFR2 Agonist-Treated Tregs and CD8+ Suppression

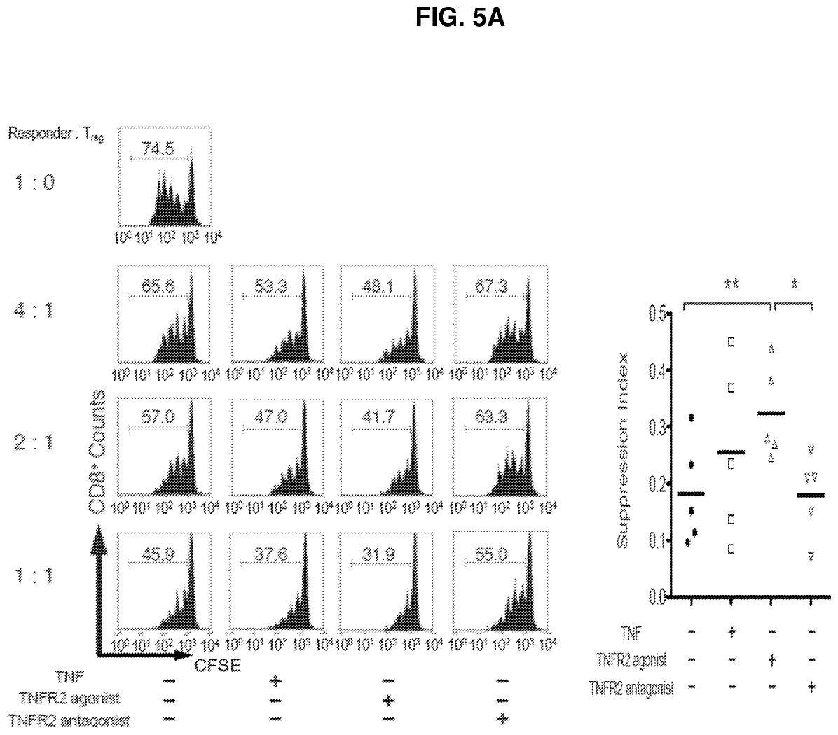

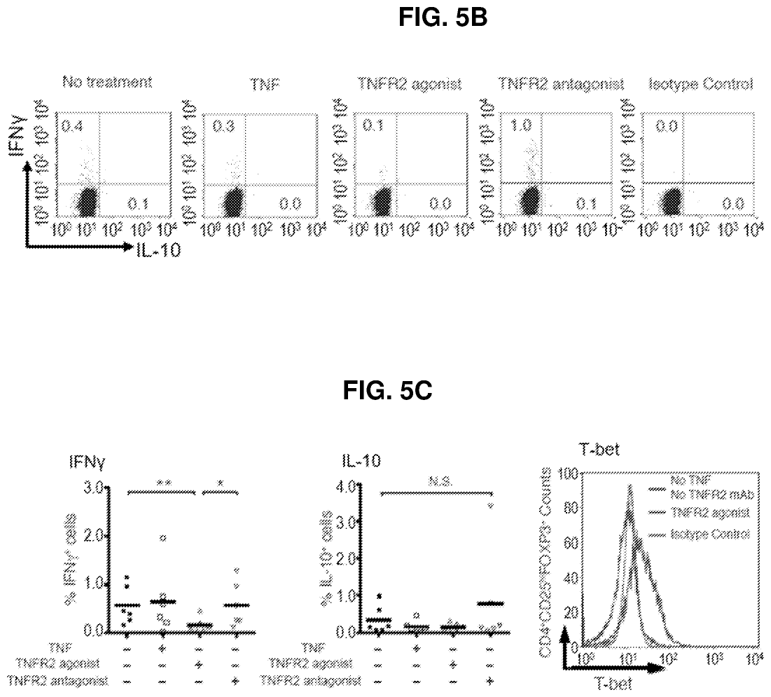

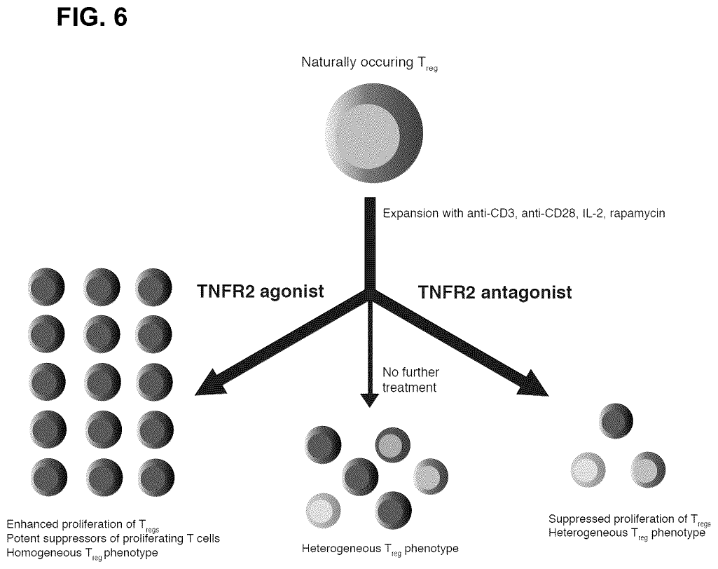

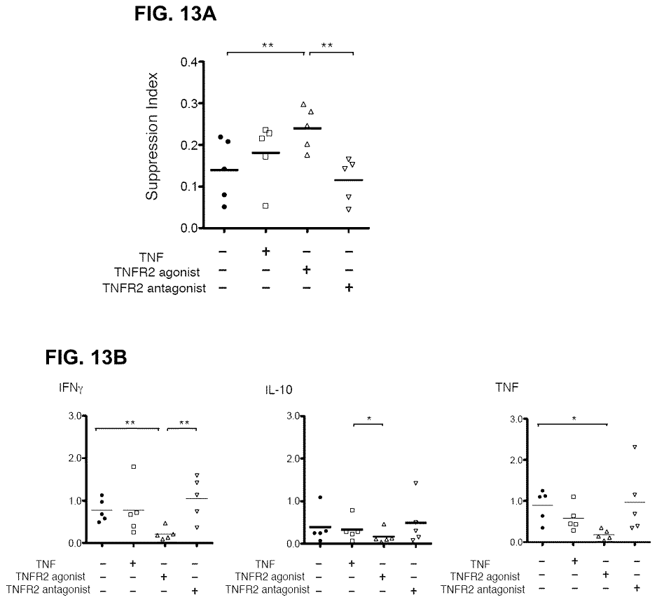

One key function of Tregs, especially in autoimmunity, is to suppress the function of autoreactive cytotoxic CD8+ T cells. I assessed this capacity by mixing Tregs from each treatment group with CFSE-stained autologous PBMC, after having stimulated them with anti-CD3mAb and IL-2 for 4 days. Autologous CD8+ T cells, the responder cells, were tested for suppression by observing the ratios of responders to Tregs. Ratios of dilution enable study of dose-dependence. All groups of Tregs displayed suppressive function on CD8+ T cells, but the degree varied by treatment group (FIG. 5A, left panel). TNFR2 agonist-treated Tregs, for example, showed the strongest suppressive capacity at 1:1 ratio (by leaving the fewest number of CD8+ cells) and then became progressively weaker at higher ratios. However, the antagonist-treated cells displayed weaker suppressive capacity that was essentially no different from that of no treatment. With a suppression index of 2:1, the TNFR2 agonist-treated group showed greater suppression than did the antagonist and no treatment groups (FIG. 5A, right panel). Similar results were observed with Tregs treated without rapamycin (FIG. 13A). The results are consistent with known phenotypes and expansion capacity of functional Tregs.

### Example 5: TNFR2 Agonist-Treated Tregs and Cytokine Production

I found that all treatment groups had relatively limited ability to produce intracellular IFNγ and IL-10 after PMA and ionomycin stimulation. But TNFR2 agonist and the TNF only-treated Tregs produced the lowest percentages of IFNγ+ cells (FIG. 5B, lowest left panel). The antagonist-treated Tregs showed significantly higher IFNγ production than the agonist (FIG. 5b, lower left panel). In similar experiments without rapamycin, the TNFR2 agonist-treated group not only produced lower IFNγ, but also lower IL-10 and TNF production relative to TNF or no treatment, respectively (FIG. 13B). Agonist-treated Tregs also showed the fewest number of TH1 Transcription Factor (T-bet)+ Tregs (FIG. 5C), which is consistent with lower IFNγ production (FIG. 5C). One of the reasons for these Tregs showing high suppression capacity over CD8+ T cells may be due to Tregs lacking the ability to produce IFNγ.

### OTHER EMBODIMENTS

The disclosures of U.S. Provisional Patent Application No. 61/762,136, filed on Feb. 7, 2013, and U.S. Provisional Patent Application No. 61/763,217, filed on Feb. 11, 2013, are hereby incorporated by reference in their entirety.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

All publications and patent applications mentioned in this specification are herein incorporated by reference to the same extent as if each independent publication or patent application was specifically and individually indicated as being incorporated by reference in their entirety.

Other embodiments are in the claims.

1-90. (canceled)

91. An isolated human, humanized or chimeric antibody or antigen-binding fragment thereof that selectively binds to an epitope of tumor necrosis-factor receptor 2 (TNFR2) that is bound by antibody clone MAB726 or antibody clone M1, wherein the antibody or antigen-binding fragment thereof has an antagonistic effect upon binding.

92. The antibody or antigen-binding fragment thereof of claim 91, wherein the antibody or antigen-binding fragment thereof is selected from the group consisting of a monoclonal antibody or antigen-binding fragment thereof, a polyclonal antibody or antigen-binding fragment thereof, a Fab, a bispecific antibody or antigen-binding fragment thereof, a monovalent antibody or antigen-binding fragment thereof, a single-chain Fv molecule, a bispecific single chain Fv ((scFv′)₂) molecule, a domain antibody, a diabody, a triabody, an affibody, a domain antibody, a small modular immunopharmaceutical (SMIP), a nanobody, a Fv fragment, a Fab fragment, a F(ab′)₂ molecule, and a tandem scFv (taFv) fragment.

93. The antibody or antigen-binding fragment thereof of claim 91, wherein the antibody or antigen-binding fragment binds an epitope of TNFR2 within amino acid positions 135-153 of SEQ ID NO: 1.

94. A pharmaceutical composition comprising the antibody or antigen-binding fragment thereof of claim 91 and a pharmaceutically acceptable carrier or excipient.

95. The pharmaceutical composition of claim 94, further comprising an anti-cancer agent, an anti-bacterial agent, and/or an anti-mycotic agent.

96. A method of treating a proliferative disorder or an infectious disease in a subject comprising administering to the subject the pharmaceutical composition of claim 94.

97. The method of claim 96, wherein the proliferative disorder is a cancer selected from the group consisting of acute lymphoblastic leukemia, acute myeloid leukemia, adrenocortical carcinoma, acquired immunodeficiency syndrome (AIDS)-related lymphoma, AIDS-related malignancies, anal cancer, astrocytoma, bile duct cancer, bladder cancer, bone cancer, osteosarcoma/malignant fibrous histiocytoma, brain stem glioma, visual pathway and hypothalamic glioma, breast cancer, bronchial adenomas/carcinoids, chronic lymphocytic leukemia, chronic myelogenous leukemia, chronic myeloproliferative disorders, clear cell sarcoma of tendon sheaths, colon cancer, colorectal cancer, cutaneous T-cell lymphoma, endometrial cancer, epithelial cancer, esophageal cancer, Ewing's family of tumors, extracranial germ cell tumor, extragonadal germ cell tumor, extrahepatic bile duct cancer, eye cancer, intraocular melanoma, retinoblastoma, gallbladder cancer, gastric cancer, hairy cell leukemia, head and neck cancer, hepatocellular cancer, Hodgkin's lymphoma, hypopharyngeal cancer, Kaposi's sarcoma, kidney cancer, laryngeal cancer, pituitary cancer, plasma cell neoplasm/multiple myeloma, pleuropulmonary blastoma, skin cancer, small cell lung cancer, small intestine cancer, soft tissue sarcoma, squamous neck cancer, testicular cancer, thyroid cancer, urethral cancer, uterine sarcoma, and vaginal cancer, or wherein the proliferative disorder is a solid tumor of the brain, lung, breast, lymphoid, gastrointestinal tract, genitourinary tract, pharynx, prostate, or ovary.

98. The method of claim 96, wherein the infectious disease is caused by:
(a) bacteria belonging to a genus selected from the group consisting of Salmonella, Streptococcus, Bacillus, Listeria, Corynebacterium, Nocardia, Neisseria, Actinobacter, Moraxella, Enterobacteriacece, Pseudomonas, Escherichia, Klebsiella, Serratia, Enterobacter, Proteus, Shigella, Yersinia, Haemophilus, Bordatella, Legionella, Pasteurella, Francisella, Brucella, Bartonella, Clostridium, Vibrio, Campylobacter, and Staphylococcus; or
(b) fungi belonging to a genus selected from the group consisting of Aspergillus, Candida, Malassezia, Trichosporon, Fusarium, Acremonium, Rhizopus, Mucor, Pneumocystis, and Absidia.

99. A kit comprising a composition comprising the antibody or antigen-binding fragment thereof of claim 91 and a pharmaceutically acceptable carrier or excipient.

100. The kit of claim 99, wherein the kit further comprises an anti-cancer agent, an anti-bacterial agent, and/or an anti-mycotic agent.

101. The kit of claim 99, wherein the kit further comprises instructions for administering the composition to a subject suffering from a proliferative disorder or an infectious disease.

102. The kit of claim 101, wherein the proliferative disorder is a cancer selected from the group consisting of acute lymphoblastic leukemia, acute myeloid leukemia, adrenocortical carcinoma, AIDS-related lymphoma, AIDS-related malignancies, anal cancer, astrocytoma, bile duct cancer, bladder cancer, bone cancer, osteosarcoma/malignant fibrous histiocytoma, brain stem glioma, visual pathway and hypothalamic glioma, breast cancer, bronchial adenomas/carcinoids, chronic lymphocytic leukemia, chronic myelogenous leukemia, chronic myeloproliferative disorders, clear cell sarcoma of tendon sheaths, colon cancer, colorectal cancer, cutaneous T-cell lymphoma, endometrial cancer, epithelial cancer, esophageal cancer, Ewing's family of tumors, extracranial germ cell tumor, extragonadal germ cell tumor, extrahepatic bile duct cancer, eye cancer, intraocular melanoma, retinoblastoma, gallbladder cancer, gastric cancer, hairy cell leukemia, head and neck cancer, hepatocellular cancer, Hodgkin's lymphoma, hypopharyngeal cancer, Kaposi's sarcoma, kidney cancer, laryngeal cancer, pituitary cancer, plasma cell neoplasm/multiple myeloma, pleuropulmonary blastoma, skin cancer, small cell lung cancer, small intestine cancer, soft tissue sarcoma, squamous neck cancer, testicular cancer, thyroid cancer, urethral cancer, uterine sarcoma, and vaginal cancer, or wherein the proliferative disorder is a solid tumor of the brain, lung, breast, lymphoid, gastrointestinal tract, genitourinary tract, pharynx, prostate, or ovary.

103. The kit of claim 101, wherein the infectious disease is caused by:
(a) bacteria belonging to a genus selected from the group consisting of Salmonella, Streptococcus, Bacillus, Listeria, Corynebacterium, Nocardia, Neisseria, Actinobacter, Moraxella, Enterobacteriacece, Pseudomonas, Escherichia, Klebsiella, Serratia, Enterobacter, Proteus, Shigella, Yersinia, Haemophilus, Bordatella, Legionella, Pasteurella, Francisella, Brucella, Bartonella, Clostridium, Vibrio, Campylobacter, and Staphylococcus; or
(b) fungi belonging to a genus selected from the group consisting of Aspergillus, Candida, Malassezia, Trichosporon, Fusarium, Acremonium, Rhizopus, Mucor, Pneumocystis, and Absidia.

104. The kit of claim 99, wherein the kit further comprises a device for administering the composition.


SEQUENCE LISTING (1 sequence)

SEQ ID NO: 1 - protein, 268 aa, Homo sapiens [sequence omitted]